(19) United States
(12) Patent Application Publication
(10) Pub. No.: US 2016/0349185 A1
(43) Pub. Date: Dec. 1, 2016

(54) STRIP FOR ANALYSIS AND APPARATUS AND SYSTEM USING STRIP FOR ANALYSIS

(71) Applicant: Samsung Electronics Co., Ltd., Suwon-si (KR)

(72) Inventors: Dae-wook PARK, Hwaseong-si (KR); Ji-hak KIM, Incheon (KR); Jin-kook YOON, Hwaseong-si (KR); Moon-seok JANG, Seoul (KR); Bo-seok MOON, Gunpo-si (KR); Ji-yeon HAN, Suwon-si (KR)

(21) Appl. No.: 15/132,731

(22) Filed: Apr. 19, 2016

(30) Foreign Application Priority Data
May 29, 2015 (KR) 10-2015-0076483

(51) Int. Cl. G01N 21/78; G01N 33/543; G01N 33/558

(52) U.S. Cl. CPC G01N 21/78; G01N 33/558; G01N 33/54366

(57) ABSTRACT

An analysis strip and an apparatus using the analysis strip are provided. The analysis strip includes an introduction part through which a sample including a target material is introduced, a target strip connected to the introduction part and configured to detect concentration of a target material included in the sample according to a flow of the introduced sample, and an auxiliary strip connected to the introduction part that is pre-impregnated with certain densities and configured to detect concentration of a mixed material of the target material and the pre-impregnated material according to the flow of the introduced sample, wherein the target strip and the auxiliary strip comprise a same material.

### CROSS-REFERENCE TO RELATED APPLICATION(S)

This application claims the benefit under 35 U.S.C. §119(a) of a Korean patent application filed on May 29, 2015 in the Korean Intellectual Property Office and assigned Serial number 10-2015-0076483, the entire disclosure of which is hereby incorporated by reference.

### TECHNICAL FIELD

The present disclosure relates to an analysis strip which is usable to analyze a sample, a cartridge which accommodates the strip, an analysis reader which is used to acquire target material information of the sample by using the strip for analysis.

### BACKGROUND

Methods of measuring biometric information through a sample collected from a human body are continually being developed. Urine or blood is generally used as the sample although other liquids such as sweat or tears are under development. In addition, other methods of measuring biometric information using saliva, exhaled breath, or the like are also being developed.

According to a general method of measuring biometric information by using a sample, the biometric information may be measured through a qualitative analysis by which positive or negative result is determined by confirming a reaction result of a reagent on a strip.

When biometric information is measured, many reagent reactions for various diseases are requested, and in addition, a qualitative analysis for a positive/negative reaction and a quantitative determination of determining a state level through a measured numeric value are requested.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide an analysis strip which is usable to analyze a sample, a cartridge which accommodates the strip, an analysis reader for analysis which is used to acquire information about target materials included in the sample by using the strip for analysis, an analysis apparatus, a system therefor, and a method of analyzing the target materials.

Another aspect of the present disclosure is to provide an analysis strip that is used to improve accuracy of a quantitative analysis of target materials included in a sample, and a quantitative analysis method.

Additional aspects will be set forth in part in the description which follows and, in part, will be apparent from the description, or may be learned by practice of the presented embodiments.

In accordance with an aspect of the present disclosure, an analysis strip is provided. The analysis strip includes an introduction part through which a sample including a target material is introduced, a target strip connected to the introduction part and configured to detect concentration of a target material included in the sample according to a flow of the introduced sample, and an auxiliary strip connected to the introduction part that is pre-impregnated with certain densities and configured to detect concentration of a mixed material of the target material and the pre-impregnated material according to the flow of the introduced sample, wherein the target strip and the auxiliary strip comprise a same material.

The reference material may include the same material as the target material.

One end of the target strip and one end of the auxiliary strip may be connected to the introduction part.

The analysis strip may further include an absorption part configured to absorb the sample, wherein the other end of the target strip and the other end of the auxiliary strip may be connected to the absorption part.

The auxiliary strip may include first and second auxiliary strips having different impregnated concentrations of the reference material.

The first and second auxiliary strips may be spaced apart from each other by interposing the target strip therebetween.

A location of the reference material impregnated in the first auxiliary strip may correspond to a location of a second reference material impregnated in the second auxiliary strip.

Each of the target strip and the auxiliary strip may have a conjugating region in which a conjugating body bondable with the target material is impregnated.

The reference material may be impregnated between the introduction part and the conjugating region.

The reference material may be impregnated in the conjugating region.

The introduction part, at least a partial region of the target strip, and at least a partial region of the auxiliary strip may be a pad of the same material.

The reference material may be impregnated in the pad.

Each of the target strip and the auxiliary strip may have a test region from which the target material is detected.

A location of the test region in the target strip may correspond to a location of the test region in the auxiliary strip.

The test region may be color-developed by at least one of the target material and the reference material.

A degree of color development of the test region may be proportional to an amount of at least one of the target material and the reference material.

The degree of color development may include a color development density.

In accordance with another aspect of the present disclosure, a cartridge for analysis is provided. The cartridge includes an analysis strip. The analysis strip comprises an introduction part through which a sample is introduced, a target strip connected to the introduction part and configured to detect concentration of a target material included in the sample, an auxiliary strip connected to the introduction part that is pre-impregnated with certain densities and configured to detect a concentration of a mixed material of the target material and the pre-impregnated material according to a flow of the sample, and a housing configured to enclose the analysis strip, wherein the target strip and the auxiliary strip comprise a same material.

In accordance with another aspect of the present disclosure, an analysis apparatus is provided. The analysis apparatus includes a light-receiver configured to receive optical information from an analysis strip comprising a target strip and first and second auxiliary strips having a target material pre-impregnated with different densities and a controller configured to determine target material information based on the optical information, a first information of the target materials pre-impregnated in the first auxiliary strip, and a second information of the target materials pre-impregnated in the second auxiliary strip.

The optical information may include first optical information corresponding to the target strip in the analysis strip and second optical information corresponding to the auxiliary strip in the strip for analysis.

The controller may be further configured to separate the first optical information and the second optical information from the optical information by using a relative location relationship of the target strip and the auxiliary strip and acquire information about the target material by using the first optical information and the second optical information.

The controller may be further configured to use a lookup table in which the optical information matches concentration information when the information about the target material is acquired.

The analysis apparatus may further include an output unit configured to output the information about the target material.

The output unit may be further configured to output compensated information of the target material as at least one of a text and a graph.

The light-receiver may include an image sensor.

The analysis apparatus may further include a light source configured to emit light onto the strip for analysis.

In accordance with another aspect of the preset disclosure, an analysis reader is provided. The analysis reader includes a light source configured to emit light onto an analysis strip for analysis, and a light-receiver configured to acquire optical information corresponding to the analysis strip, wherein the analysis strip includes a target strip and an auxiliary strip having a pre-impregnated target material.

The light-receiver may be further configured to acquire first optical information corresponding to the target strip in the analysis strip and second optical information corresponding to the auxiliary strip in the strip for analysis.

The analysis reader may further include a communication unit configured to transmit the optical information to an external device.

In accordance with another aspect of the present disclosure, a system for analysis is provided. The system for analysis includes the reader for analysis, and an analysis apparatus configured to acquire information about the target material by using the optical information.

The analysis apparatus may be further configured to use a lookup table in which the optical information matches concentration information when the information about the target material is acquired.

The analysis apparatus may include an output unit configured to output the information about the target material.

In accordance with another aspect of the present disclosure, a target material analysis method is provided. The target material analysis method includes acquiring optical information of the strip for analysis, and acquiring information about target material introduced into the analysis strip by using the optical information.

The acquiring of the optical information may include receiving light outputted from the strip for analysis.

The acquiring of the optical information may include emitting light onto the strip for analysis.

The acquiring of the information about the target material may include separating first optical information corresponding to the target strip and second optical information corresponding to the auxiliary strip and acquiring the information about the target material by using the first optical information and the second optical information.

When the information about the target material is acquired, a lookup table in which the optical information matches concentration information may be used.

The target material analysis method may further include outputting the information about the target material.

The target material analysis method may further include outputting information about a health state corresponding to the information about the target material.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates a perspective view of an analysis strip according to an embodiment of the present disclosure;

FIG. 2 illustrates a top view of the analysis strip of FIG. 1 according to an embodiment of the present disclosure;

FIG. 3 illustrates a cross-sectional view of the analysis strip of FIG. 1 according to an embodiment of the present disclosure;

FIG. 4 illustrates a scanning electron microscope (SEM) image of nitrocellulose applicable to an analysis strip according to an embodiment of the present disclosure;

FIG. 5 illustrates a diagram for describing an operation in which a target material passes through a target strip according to an embodiment of the present disclosure;

FIG. 6 illustrates a diagram for describing an operation in which a target material passes through an auxiliary strip according to an embodiment of the present disclosure;

FIGS. 7 and 8 illustrate top views of analysis strips according to various embodiments of the present disclosure;

FIGS. 9 to 11 illustrate cross-sectional views of analysis strips according to various embodiments of the present disclosure;

FIGS. 12 and 13 illustrate top views of analysis strips according to various embodiments of the present disclosure;

FIG. 14 illustrates a diagram for describing a method of using an analysis strip according to an embodiment of the present disclosure;

FIG. 15 illustrates an analysis strip according to an embodiment of the present disclosure;

FIG. 16 illustrates an exploded perspective view of a cartridge including an analysis strip according to an embodiment of the present disclosure;

FIG. 17 illustrates an outer appearance perspective view of the cartridge of FIG. 16 according to an embodiment of the present disclosure;

FIGS. 18A and 18B illustrate an analysis strip attached to wearable items according to an embodiment of the present disclosure;

FIG. 19 illustrates a diagram for describing a band-type cartridge according to an embodiment of the present disclosure;

FIG. 20 illustrates a diagram for describing an analysis strip which is attachable to and detachable from a smartwatch according to an embodiment of the present disclosure;

FIG. 21 illustrates a diagram for describing a method by which the smartwatch of FIG. 20 displays an analysis result according to an embodiment of the present disclosure;

FIG. 22 illustrates a block diagram of an analysis system according to an embodiment of the present disclosure;

FIG. 23 illustrates a block diagram of an analysis reader in the analysis system of FIG. 22 according to an embodiment of the present disclosure;

FIG. 24 illustrates a block diagram of an analysis apparatus in the analysis system of FIG. 22 according to an embodiment of the present disclosure;

FIG. 25A illustrates a cross-sectional view of an analysis reader according to an embodiment of the present disclosure;

FIG. 25B illustrates a cross-sectional view of the analysis reader of FIG. 25A in another point of view according to an embodiment of the present disclosure;

FIG. 26 illustrates a diagram of a reflective reader according to an embodiment of the present disclosure;

FIG. 27 is a flowchart of a method for analyzing a sample according to an embodiment of the present disclosure;

FIG. 28 is a flowchart for acquiring target material information according to an embodiment of the present disclosure;

FIG. 29 illustrates a graph of a correlation between optical information and concentration information according to an embodiment of the present disclosure;

FIG. 30 illustrates a graph of target material information according to an embodiment of the present disclosure;

FIG. 31 illustrates a block diagram of an analysis system according to an embodiment of the present disclosure;

FIG. 32 illustrates an outer appearance of the analysis system of FIG. 31 according to an embodiment of the present disclosure;

FIG. 33 illustrates an optical structure of the analysis system of FIG. 31 according to an embodiment of the present disclosure;

FIG. 34 illustrates an outer appearance of an analysis apparatus according to an embodiment of the present disclosure;

FIG. 35 illustrates a block diagram of the analysis apparatus of FIG. 34 according to an embodiment of the present disclosure;

FIG. 36 illustrates a block diagram of an analysis apparatus which may perform functions besides a diagnosis application according to an embodiment of the present disclosure;

FIG. 37 illustrates a diagram for describing a setup mode of a diagnosis application according to an embodiment of the present disclosure;

FIG. 38 illustrates a diagram for describing a method of determining a diagnosis item according to an embodiment of the present disclosure;

FIG. 39 illustrates a diagram for describing a method of providing a diagnosis item according to an embodiment of the present disclosure;

FIG. 40 illustrates a diagram for describing a method of setting a detailed plan of a diagnosis item according to an embodiment of the present disclosure;

FIG. 41 illustrates a diagram for describing a method of changing a detailed plan according to an embodiment of the present disclosure;

FIG. 42 is a flowchart of a method for executing a diagnosis according to an embodiment of the present disclosure;

FIG. 43 illustrates a diagram for describing an alarm for a diagnosis according to an embodiment of the present disclosure;

FIG. 44 illustrates a diagram for describing a method of providing a diagnosis result, according to an embodiment of the present disclosure;

FIG. 45 illustrates a diagram for describing a method of providing a diagnosis result according to an embodiment of the present disclosure; and

FIG. 46 illustrates a diagram for describing a network supporting a medical service according to an embodiment of the present disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

An analysis strip according to an embodiment may quantitatively analyze a target material by using immunochromatography.

A sample analyzable using the analysis strip according to an embodiment may be a fluid or a material having similar liquidity to a fluid, which includes or does not include the target material to be analyzed. The sample introduced into the analysis strip may undergo preprocessing for modifying or changing characteristics of the sample or may be directly obtained from a source.

The source of the sample may be a biological source such as blood, an interstitial fluid, saliva, an ocular lens fluid, a cerebral spinal fluid, sweat, urine, an ascites fluid, raucous, a synovial fluid, a peritoneal fluid, a vaginal fluid, an amniotic fluid, or a physiological fluid including a material similar thereto. However, the source of the sample is not limited to bodily fluids. The source of the sample may be an environmental sample for water quality management or soil management, for example.

The target material included in the sample is a compound to be analyzed and may be referred to as a marker. For example, the target material may be nucleic acid, high sensitivity C-reactive protein (hsCRP), a micro C-reactive protein (microCRP), glycated hemoglobin (HbA1c), microalbumin, prostate specific antigen (PSA), alpha-fetoprotein (AFP), cardiac troponin I (cTnI), glucose, C-reactive protein (CRP), or the like, and types of the target material are not limited.

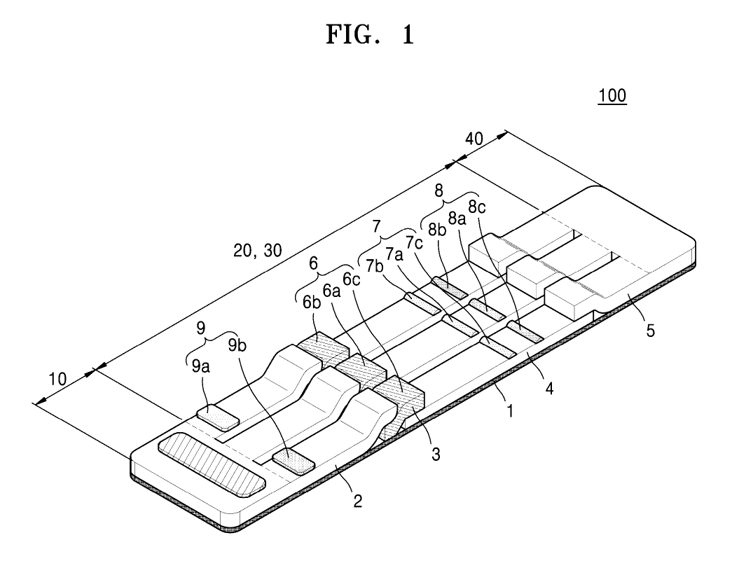

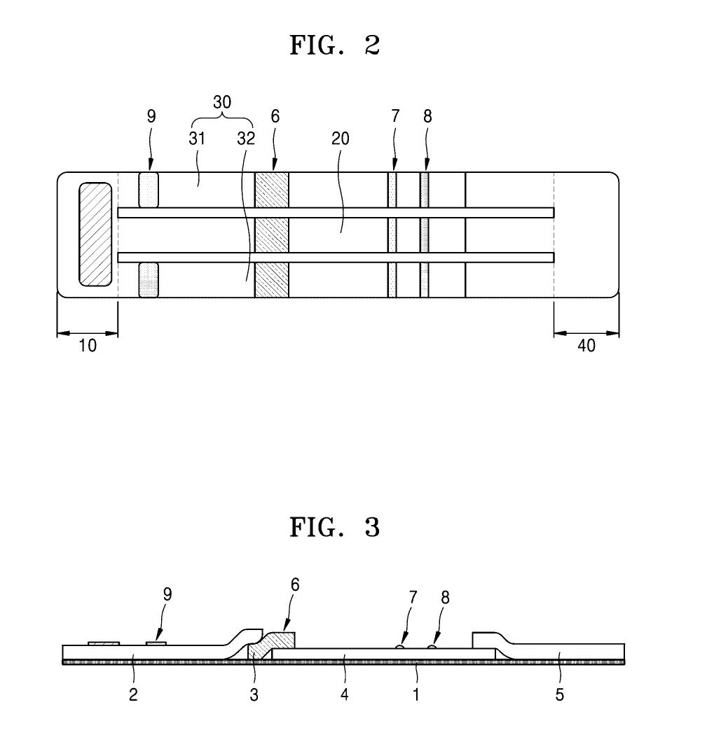

FIG. 1 illustrates a perspective view of an analysis strip according to an embodiment of the present disclosure, FIG. 2 illustrates a top view of the analysis strip of FIG. 1 according to an embodiment of the present disclosure, and FIG. 3 illustrates a cross-sectional view of the analysis strip of FIG. 1 according to an embodiment of the present disclosure.

Referring to FIGS. 1 to 3, an analysis strip 100 (hereinafter, “analysis strip 100” or “strip”) may include a base member 1 and a loading pad 2, a conjugating pad 3, a membrane 4, and an absorption pad 5 which are sequentially arranged on the base member 1 in a first direction, e.g., a lengthwise direction. The strip may indicate a material piece cut with a necessary width from a sheet.

If a sample is introduced into the loading pad 2, the sample flows due to a capillary phenomenon. Accordingly, the sample may move along the conjugating pad 3 and the membrane 4 and end the flow by being absorbed by the absorption pad 5. In this process, a target material in the sample may be bonded with a certain nucleic acid or antibody that is bonded to the conjugating pad 3, and non-bonded materials may continuously move with the sample and be absorbed by the absorption pad 5.

The base member 1 may support the other components of the strip, e.g., the loading pad 2, the conjugating pad 3, the membrane 4, and the absorption pad 5. The base member 1 may include a material having at least one characteristic of water insolubility, non-porosity, and rigidity. For example, the base member 1 may include polyethylene, polyester, polypropylene, poly(4-methylbutane), polystyrene, polymethacrylate, poly(ethylene terephthalate), nylon, poly(vinyl butyrate), glass, ceramic, metal, or the like but is not limited thereto.

A length of the base member 1 may be equal to or longer than a connected length of the loading pad 2, the membrane 4, and the absorption pad 5 arranged on the base member 1.

Although the base member 1 is shown in FIGS. 1 to 3, the analysis strip 100 according to an embodiment may not include the base member 1. For example, the analysis strip 100 may include only the loading pad 2, the conjugating pad 3, the membrane 4, and the absorption pad 5 sequentially arranged in the first direction, and in this case, the membrane 4 may act as the base member 1.

Each of the loading pad 2, the conjugating pad 3, the membrane 4, and the absorption pad 5 may contact in at least a partial region with a neighboring pad. The term “contacting” used in the present application indicates connecting or overlapping parts such that a flow of a sample due to a capillary phenomenon occurs between the parts. For example, one end of the conjugating pad 3 may overlap the loading pad 2, and the other end of the conjugating pad 3 may overlap the membrane 4. In addition, one end of the membrane 4 may overlap the conjugating pad 3, and the other end of the membrane 4 may overlap the absorption pad 5. Alternatively, although not shown, the one end of the conjugating pad 3 may connect to the loading pad 2, and the other end of the conjugating pad 3 may connect to the membrane 4.

In addition, the distal end of the membrane 4 may also connect to an end of the absorption pad 5. Alternatively, the conjugating pad 3 may overlap any one of the loading pad 2 and the membrane 4, which are adjacent to the conjugating pad 3, and connect to the other one. The membrane 4 may also overlap any one of the conjugating pad 3 and the absorption pad 5, which are adjacent to the membrane 4, and connect to the other one. Even when the end portions are connected to or overlap each other, the sample may flow to a neighboring pad due to a capillary phenomenon.

The loading pad 2, the conjugating pad 3, the membrane 4 may include a material having gas pockets through which the sample spreads due to a capillary phenomenon. For example, the loading pad 2, the conjugating pad 3, the membrane 4, and the absorption pad 5 may include fibrous paper, a paper-like microporous membrane, cellulose, a cellulose derivative (e.g., cellulose acetate), nitrocellulose, a fabric (e.g., a glass fiber, natural cotton, or nylon), porous gel, or the like but is not limited thereto.

A proximal end of the loading pad 2 may load the sample, and a distal end of the loading pad 2 may contact a proximal end of the conjugating pad 3.

The loading pad 2 may filter insoluble particles and impurities in the sample. For example, if the loading pad 2 is formed with a cellulose-based filter bed or a glass fiber filter bed, the loading pad 2 may spread and filter the sample.

In addition, the loading pad 2 may be preprocessed to prevent a target material, e.g., nucleic acid, in the sample from being absorbed, assist ingredients of the sample to easily move, and maintain sensitivity of a reaction. For example, the loading pad 2 may be preprocessed by inert proteins or a surfactant. The preprocessing may be determined according to types of a target material and a sample and the loading pad 2 may be vacuum-dried at a high temperature.

A proximal end of the conjugating pad 3 may contact a distal end of the loading pad 2, and a distal end of the conjugating pad 3 may contact a proximal end of the membrane 4. The conjugating pad 3 may include a material having gas pockets and accommodate the sample from the loading pad 2.

A conjugating body that is bondable with the target material in the sample may be impregnated into the conjugating pad 3. For example, a permeating a material liquefied from a gas state into an object is impregnated to treat the target material with preservatives, damp-proofing the target material, dyeing the target material, and the like. The conjugating body may include a detector (e.g., a secondary antibody such as a detection antibody) that is attached to the target material and a color development particle. The detector and the color development particle in the conjugating body may be bonded in a mutually conjugated state. When the sample applied spreads to the conjugating pad 3, and the target material in the sample may be bonded with the detector in the conjugating body, thereby forming a first complex.

The proximal end of the membrane 4 may contact the conjugating pad 3, and the distal end of the membrane 4 may contact a proximal end of the absorption pad 5. The membrane 4 may be disposed on the base member 1 but is not limited thereto. The membrane 4 may become the base member 1. The membrane 4 may also include a material having gas pockets and accommodate the sample spread and moved from the conjugating pad 3. The gas pockets in the membrane may have a higher density or a lower density than, for example, the conjugating pad 3.

The membrane 4 may include a test region 7 from which the target material is detected and a control region 8 from which the conjugating body is detected. For example, a plurality of first captors (e.g., primary antibody such as a capture antibody) may be attached to and bonded with the target material in the sample and disposed in the test region 7 in a fixed state. In such an example, a plurality of second captors bonded with the detector of the conjugating body may be disposed in the control region 8 in a fixed state. The test region 7 and the control region 8 may be formed in a line shape crossing a lengthwise direction of the membrane 4. The test region 7 and the control region 8 may penetrate through the membrane 4 in a widthwise direction of the membrane 4.

In the test region 7, the first captors fixed in a spot form may be arranged over a certain section of the membrane 4. The plurality of first captors may be arranged one-dimensionally or two-dimensionally. In the control region 8, the second captors fixed in a spot form may also be arranged. The plurality of second captors may also be arranged one-dimensionally or two-dimensionally.

The detector in the conjugating pad 3 may move along with the flow of the sample, whereas the first and second captors of the membrane 4 may be fixed in the test region 7 or the control region 8 without moving along with the flow of the sample.

When the sample is introduced from the conjugating pad 3, the sample may be selectively bonded with the first captors in the test region 7 while moving along the membrane 4 in the lengthwise direction by a capillary flow. For example, the target material in the sample may become a first complex by being bonded with the conjugating body in the conjugating pad 3 through an antigen-antibody reaction, and the first complex may become a second complex according to the sandwich assay principle by being bonded with the first captor in the test region 7. Thereafter, the second complex may be fixed in the test region 7. The first captor is color-developed by a first color development particle of the first complex.

When the first captors are two-dimensionally arranged, the first complex may move along the membrane 4 and then color-develop the first captor in contact with a first capture line of the test region 7, and according to concentration of the target material in the sample, the first captor in a next capture line is color-developed.

For example, when concentration of the target material in the sample is relatively low, all of the first complexes in the sample may be bonded with the first captors in the first capture line and cannot be bonded with the first captors in a second capture line. Accordingly, only the first capture line may be color-developed. When concentration of the target material in the sample is relatively high, first complexes remaining after color-developing the first capture line of the test region 7 may sequentially color-develop by the next capture lines. As described above, concentration of the target material may be detected based on the number of color-developed capture lines or color-developed spots. That is, a quantitative analysis on the target material may be performed by measuring a density of color-developed first captors.

An amount of the conjugating body permeated into the conjugating pad 3 may be greater than an amount of the target material estimated to be included in the sample. The conjugating body that is not bonded with the target material in the sample in the conjugating pad 3 may pass through the test region 7 as the sample flows along the membrane 4.

The conjugating body which has passed through the test region 7 may be attached to the second captor to be specifically bonded with the detector of the conjugating body while flowing through the control region 8. In the membrane 4, the second captor which is not attached to the target material but is specifically attached to the detector of the conjugating body may be fixed in the control region 8. The second captor does not flow with the sample by being fixed in the control region 8.

The second captor in the control region 8 indicates whether the sample and the conjugating body have moved into the control region 8 due to a capillary phenomenon regardless of whether the target material exists in the sample, and indicates whether a capillary phenomenon has occurred, and determines a validity of a measurement result.

For example, if the second captor in the control region 8 is not color-developed by a first color-development particle of the conjugating body, it may be determined that the analysis strip 100 does not normally operate. When the second captor is color-developed by being bonded with the conjugating body in the sample, target information including the presence/absence and concentration of the target material may indicate that the data is valid.

In addition, the test region 7 may not be color-developed when the control region 8 is color-developed. In this case, a user may determine that the target material does not exist in the sample. In addition, the test region 7 may be color-developed when the control region 8 is not color-developed. In this case, the user may determine that the target material exists in the sample, and concentration of the target material may be determined. It may be determined that the concentration of the target material is high when a color development density is high.

A spaced arrangement may be performed as described below. The test region 7 and the control region 8 may be sequentially spaced and arranged in a direction from the conjugating pad 3 to the absorption pad 5. However, the spaced arrangement is not limited thereto. The control region 8 and the test region 7 may be sequentially spaced and arranged in the direction from the conjugating pad 3 to the absorption pad 5.

The sample, which has passed through the membrane 4, may flow into and be absorbed by the absorption pad 5 disposed at the distal end of the base member 1. The absorption pad 5 may physically absorb the sample and absorb non-reacted materials. For example, the absorption pad 5 may be manufactured to absorb about 70% to 85% of a total amount of the sample introduced into the strip. A length of the absorption pad 5 may be based on an amount of a sample, absorption capability thereof, a moving time of the sample, and the like. The absorption pad 5 may act as a pump or storage for adjusting a moving speed of the sample or containing the sample. The moving speed of the sample may vary quality and a size of the absorption pad 5.

The absorption pad 5 may include nitrocellulose, cellulose ester, glass (e.g., borosilicate glass fiber), polyether sulfone, cotton, dehydrated polyacrylamide, silica gel, polyethylene glycol, or the like but is not limited thereto.

In addition, one or more second color development particles may be disposed in the absorption pad 5 and may be configured to be color-developed in response to absorption of a sample. The second color development particle may be a material of which a color is changed by being bonded with a certain material of a sample, e.g., water. The second color development particles may be arranged at a location where a sample is introduced with sufficient volume to detect the target material. Alternatively, the second color development particles may be one-dimensionally or two-dimensionally arranged in a spot form, and an amount of an introduced sample may be determined based on the number or a location of second color development particles.

The loading pad 2, the conjugating pad 3, the membrane 4, and the absorption pad 5 described above may be assembled by an adhesive and fixed to the base member 1 by the adhesive. The adhesive may be a pressure-sensitive adhesive (PSA) but is not limited thereto. The pads described above may be assembled by permeating the adhesive into gas pockets of the pads and accordingly assembling the pads with the base member 1.

The analysis strip 100 shown in FIGS. 1 to 3 includes the loading pad 2, the conjugating pad 3, the membrane 4, and the absorption pad 5 but is not limited thereto.

The analysis strip 100 may include an introduction part 10 for receiving a sample including a target material from the outside, a target strip 20 for receiving the sample that flows from the introduction part 10 (hereinafter, referred to as “first sample”), an auxiliary strip 30 which is spaced apart from the target strip 20, in which a reference material is impregnated, in which a portion of the sample that flows from the introduction part 10 (hereinafter, referred to as “second sample”) to detect the reference material and the target material, and an absorption part 40 configured to absorb the sample that flows through the target strip 20 and the auxiliary strip 30.

The introduction part 10 may receive a sample such as a liquid including the target material (e.g., blood, a tissue liquid, a lymph fluid, bone marrow, saliva, urine, or the like) but is not limited thereto. The target material is a compound to be analyzed in the sample and is also called a marker. For example, the target material may be nucleic acid or CRP. A portion of the loading pad 2 may be the introduction part 10.

Since the target strip 20 and the auxiliary strip 30 are spaced apart from each other, samples flowing in the target strip 20 and the auxiliary strip 30 may not be mixed with each other. For example, the membrane 4, the conjugating pad 3, the absorption pad 5, and the loading pad 2 may be sequentially assembled on the base member 1, and openings may be formed so as to penetrate through the conjugating pad 3 and the membrane 4 in the lengthwise direction of the analysis strip 100 and separate partial regions of the loading pad 2 and the absorption pad 5. The openings may be shaped as a rectangle that is long in the lengthwise direction of the analysis strip 100. The target strip 20 and the auxiliary strip 30 may have different proximal ends to receive different portions of the sample from the introduction part.

A proximal end of the target strip 20 may be connected to a distal end of the introduction part 10, and the distal end of the target strip 20 may be connected to a proximal end of the absorption part 40. The target strip 20 may include a conjugating region 6a in which the conjugating body is permeated, a test region 7a in which the first captor is fixed, and a control region 8a in which the second captor is fixed. That is, the conjugating pad 3 may be the conjugating region 6a of the target strip 20. In addition, the test region 7a and the control region 8a of the target strip 20 may be formed in partial region of the membrane 4.

When the analysis strip 100 is used for a qualitative analysis, the presence/absence of the target material may be determined based on a degree of color development in the test region 7 and the control region 8. However, when the analysis strip 100 is used for a quantitative analysis, it is difficult to perform the quantitative analysis because of large variability in the analysis as set forth below.

The analysis strip 100 is formed by assembling porous pads (e.g., the loading pad 2, the conjugating pad 3, the membrane 4, and the like), which may have a non-uniform gas pocket distribution.

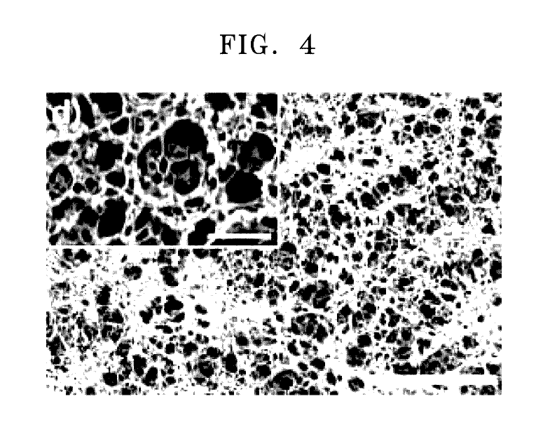

FIG. 4 illustrates a scanning electron microscope (SEM) image of nitrocellulose applicable to the analysis strip according to an embodiment of the present disclosure.

Referring to FIG. 4, a distribution of gas pockets included in the nitrocellulose is not uniform. That is, the gas pockets of a pad do not have a constant size, and gas pockets of sizes of about 8 μm to about 15 μm are randomly distributed. Since this distribution is not the same for each strip, samples including the target material of the same concentration may have a different analysis result.

In addition, amounts and uniformity of the conjugating body, the first captor, and the second captor arranged in a conjugating region 6, the test region 7, and the control region 8 may also vary for each analysis strip 100, and the conjugating body, the first captor, and the second captor may be dissolved over time after manufacturing the strip. Furthermore, a distance between pads and a strength and uniformity of the adhesive may vary for each analysis strip 100. Accordingly, it may be difficult to quantitatively analyze the analysis strip 100.

Referring back to FIGS. 1 to 3, the analysis strip 100 according to an embodiment may include the auxiliary strip 30 containing the reference material to increase a precision of a quantitative analysis on the target material. According to an embodiment, a proximal end of the auxiliary strip 30 may be connected to the introduction part 10, and the distal end of the auxiliary strip 30 may be connected to the absorption part 40. The auxiliary strip 30 may also include conjugating regions 6b and 6c in which the conjugating body is permeated, test regions 7b and 7c in which the first captor is fixed, and control regions 8b and 8c in which the second captor is fixed. That is, the conjugating pad 3 may be the conjugating regions 6b and 6c of the auxiliary strip 30. In addition, a partial region of the membrane 4 may be the test regions 7b and 7c and the control regions 8b and 8c of the auxiliary strip 30.

Locations of the conjugating regions 6b and 6c, the test regions 7b and 7c and the control regions 8b and 8c of the auxiliary strip 30 may correspond to locations of the conjugating region 6a, the test region 7a and the control region 8a of the target strip 20, respectively.

The target material of a certain density may be impregnated in the auxiliary strip 30. Hereinafter, the impregnated target material is referred to as a reference material. The auxiliary strip 30 may further include a reference region 9 in which the reference material is permeated. The reference material may include the same material as the target material. For example, when the target material is glucose, the reference material may also be glucose. The reference region 9 may be disposed between the introduction part 10 and the conjugating region 6. Although the reference region 9 is spaced apart from the conjugating region 6, the present embodiment is not limited thereto. The reference region 9 may be disposed prior to the test region 7. For example, the reference region 9 may overlap the conjugating region 6. That is, both the conjugating body and the reference material may be impregnated in the conjugating region 6.

The auxiliary strip 30 may include first and second auxiliary strips 31 and 32 in which contained densities of the reference material are different from each other. For example, if the analysis strip 100 according to an embodiment is a strip for detecting glucose as the target material, the first auxiliary strip 31 may include a reference region 9a in which glucose having a density of about 1% is impregnated, and the second auxiliary strip 32 may include a reference region 9b in which glucose having a density of about 3% is impregnated.

The first and second auxiliary strips 31 and 32 may be spaced apart from each other by interposing the target strip 20 therebetween, but are not limited thereto. The target strip 20, the first auxiliary strip 31, and the second auxiliary strip 32 may be arranged in a widthwise direction of the analysis strip 100. The more the number of auxiliary strips 30, the higher a precision of a quantitative analysis. Even though a plurality of auxiliary strips 30 are used, locations of reference regions 9 of the auxiliary strips 30 may correspond to each other.

Hereinafter, an operation in which the target material passes through the target strip 20 and the auxiliary strip 30 is described.

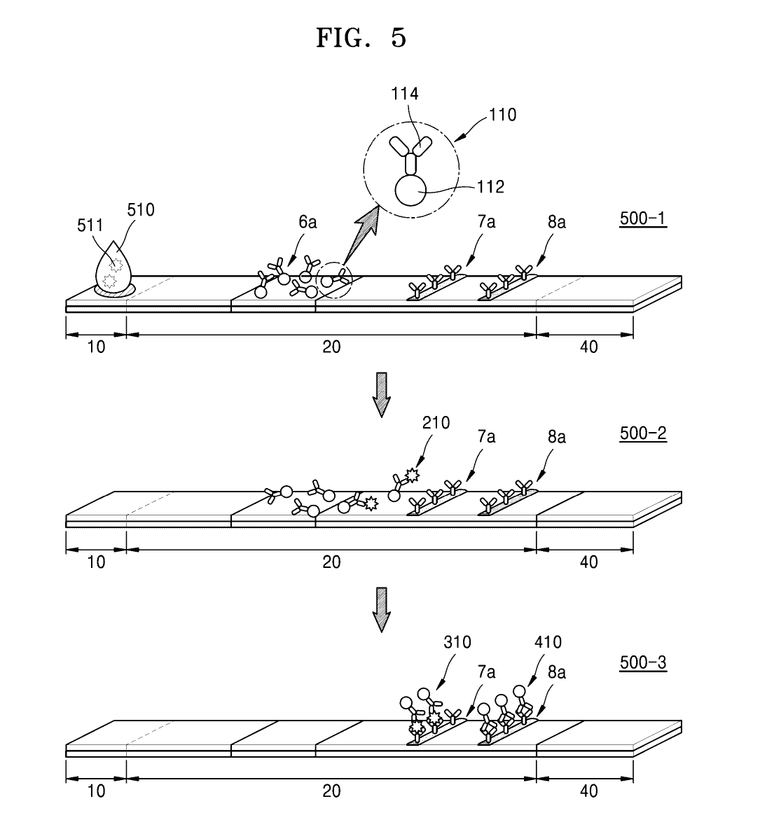

FIG. 5 illustrates a diagram for describing an operation in which a target material passes through a target strip according to an embodiment of the present disclosure.

Referring to FIG. 5, in reference numeral 500-1, a first sample 510 is introduced from the introduction part 10 may flow on the target strip 20 due to a capillary phenomenon. The first sample 510 may pass through the conjugating region 6a, the test region 7a, and the control region 8a while flowing on the target strip 20.

The first sample 510 flows into the conjugating region 6a of the target strip 20. A conjugating body 110 is impregnated in the conjugating region 6a. The conjugating body 110 may include a detector 114 (second antibody such as a detection antibody), which is specifically attached to the target material 511, and a color development particle 112. The detector 114 and the color development particle 112 may be bonded in a form conjugated with each other in the conjugating body 110. Referring to reference numeral 500-2, the first sample 510 may spread to the conjugating region 6a, and accordingly, the target material 511 in the first sample 510 may be bonded with the detector 114 in the conjugating body 110, thereby forming a first complex 210.

The first complex 210, the conjugating body 110 which is not bonded with the target material 511, and the like continuously flow with the first sample 510 into the test region 7a. Referring to reference numeral 500-3, the first sample 510 flows due to a capillary phenomenon. In the test region 7a, a first captor bonded with the first complex 210 is fixed. The first captor becomes a second complex 310 by being bonded with the first complex 210, and the test region 7a is color-developed by the color development particle 112 of the second complex 310.

The first complex 210 which is not bonded with the first captor, the conjugating body 110, and the like continuously flow with the first sample 510 into the control region 8a. The first sample 510 floats due to a capillary phenomenon. In the control region 8a, a second captor specifically bonded with the conjugating body 110 is fixed. The second captor becomes a third complex 410 by being bonded with the conjugating body 110, and the control region 8a is color-developed by color development particle 112 of the third complex 410.

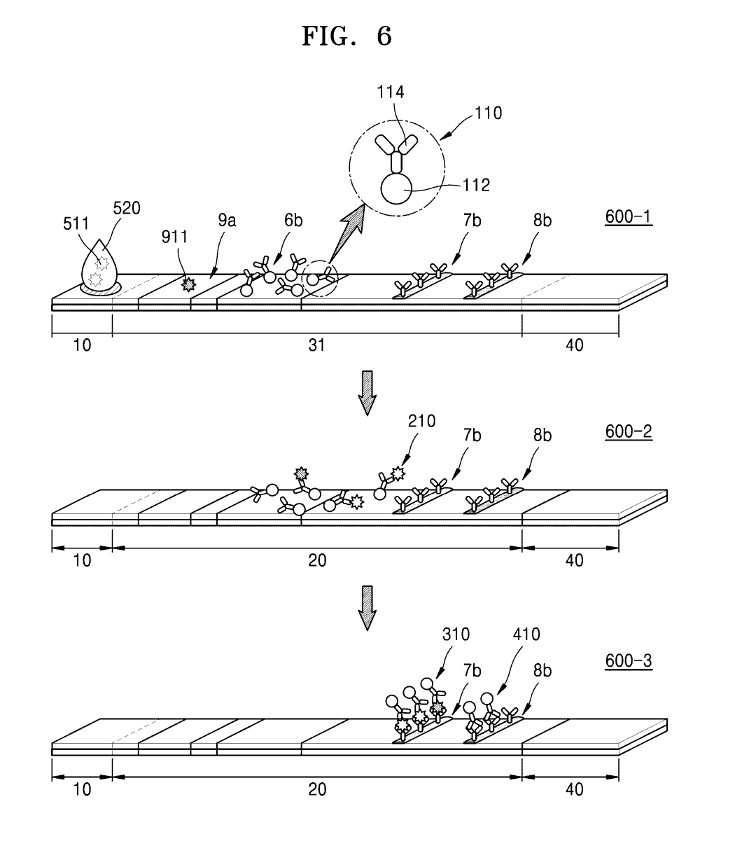

FIG. 6 illustrates a diagram for describing an operation in which a target material passes through the auxiliary strip according to an embodiment of the present disclosure.

Referring to FIG. 6, a second sample 520, which is a portion of the sample applied from the introduction part 10 and includes the target material 511, may flow on the auxiliary strip 30 due to a capillary phenomenon. As illustrated in reference numeral 600-1, the second sample 520 may pass through the reference region 9a, the conjugating region 6b, the test region 7b, and the control region 8b.

The second sample 520 flows into the reference region 9a, where a reference material is impregnated. The reference material flows due to a capillary phenomenon along with a flow of the second sample 520. The second sample 520, which includes the reference material, has a higher concentration of the target material 511 in the auxiliary strip 30 than that in the target strip 20 because the reference material includes the same material as the target materials 511. For convenience of description, the reference material and the target material 511 are distinguished from each other.

The second sample 520 flows into the conjugating region 6b. The conjugating body 110 is impregnated in the conjugating region 6b. The conjugating body 110 may include the detector 114 (second antibody such as a detection antibody) and the color development particle 112. The detector 114 attaches to the target material 511 and the reference material. The detector 114 and the color development particle 112 may be bonded in a form conjugated with each other in the conjugating body 110.

Since the reference material is the same as the target material 511, the conjugating body 110 may also be bonded with the reference material. Referring to reference numeral 600-2, the second sample 520 may flow onto the conjugating region 6b and the target material 511 and the reference material in the second sample 520 may be bonded with the detector 114 in the conjugating body 110, thereby forming the first complex 210. Since the reference material is the same as the target material 511, concentration of the first complex 210 in the second sample 520 may be higher than the concentration of the first complex 210 in the first sample 510.

The first complex 210, the conjugating body 110 which is not bonded with the target material 511, and the like continuously flow with the second sample 520 and into the test region 7b. The second sample 520 flows due to a capillary phenomenon. In the test region 7b, a first captor specifically bonded with the first complex 210 is fixed. Referring to reference numeral 600-3, the first captor becomes a second complex 310 by being bonded with the first complex 210, and the test region 7b is color-developed by the color development particle 112 of the second complex 310.

A degree of color development of the second complex 310 in the auxiliary strip 30 may be greater than a degree of color development of the second complex 310 in the target strip 20 because the second complex 310 further includes the second complex 310 due to the reference material. A precision of a quantitative analysis on the target material 511 may be increased by compensating for the degree of color development of target strip 20 based on the degree of color development of the auxiliary strip 30.

The first complex 210 which is not bonded with the first captor, the conjugating body 110, and the like continuously flow with the second sample 520 and flow into the control region 8b. The second sample 520 flows due to a capillary phenomenon. In the control region 8b, a second captor specifically bonded with the conjugating body 110 is fixed. The second captor becomes the third complex 410 by being bonded with the conjugating body 110, and the control region 8b is color-developed by color development particle 112 of the third complex 410.

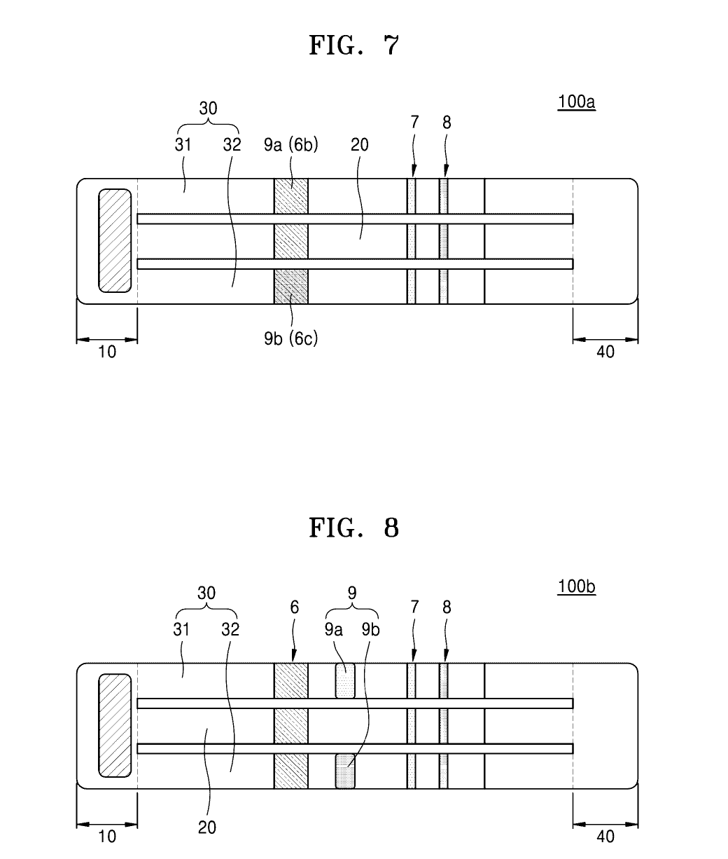

FIGS. 7 and 8 illustrate top views of analysis strips according to various embodiments of the present disclosure.

Referring to FIG. 7, the reference regions 9a and 9b of an analysis strip 100a may overlap the conjugating regions 6b and 6c. For example, the reference region 9a in which the reference material of a first density is impregnated may overlap the conjugating region 6b of the first auxiliary strip 31, and the reference region 9b in which the reference material of a second density is impregnated may overlap the conjugating region 6c of the second auxiliary strip 32. The first density may differ from the second density in terms of the impregnation density. The reference regions 9a and 9b may fully overlap the conjugating regions 6b and 6c or may partially overlap the conjugating regions 6b and 6c.

Referring to FIG. 8, in an analysis strip 100b, the reference region 9 may be disposed between the conjugating region 6 and the test region 7. Even though the reference region 9 is disposed between the conjugating region 6 and the test region 7, a location of the reference region 9a of the first auxiliary strip 31 may correspond to a location of the reference region 9b of the second auxiliary strip 32. In addition, a density of the reference material impregnated in the first auxiliary strip 31 may differ from a density of the reference material impregnated in the second auxiliary strip 32.

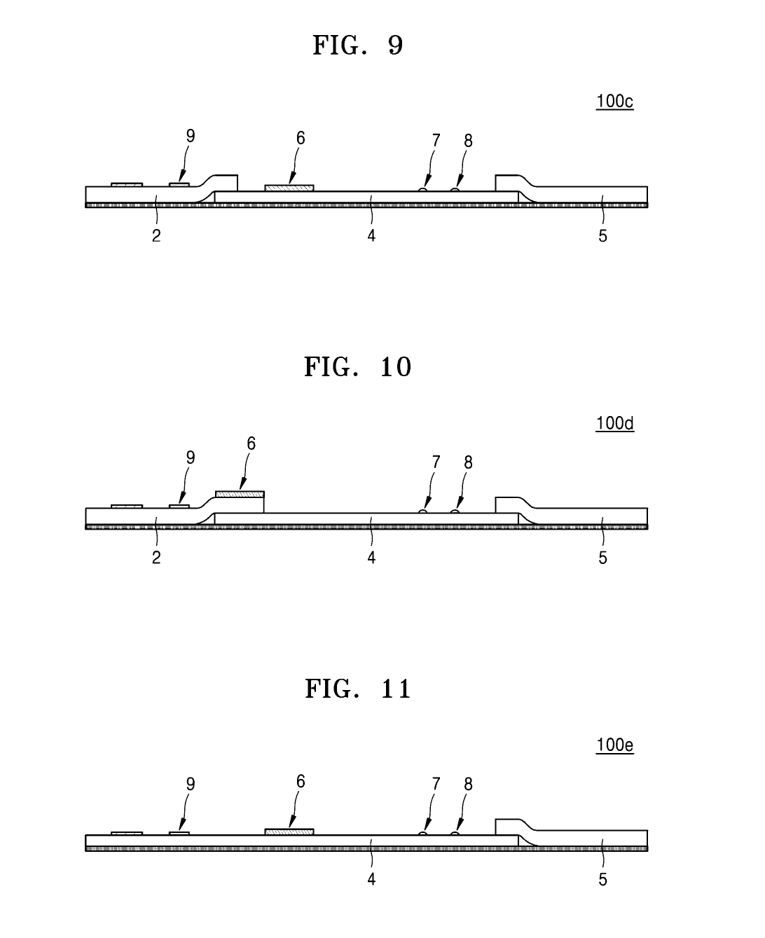

FIGS. 9 to 11 illustrate cross-sectional views of analysis strips according to various embodiments of the present disclosure.

Referring to FIG. 9, an analysis strip 100c may include the loading pad 2, the membrane 4, and the absorption pad 5 sequentially arranged in a lengthwise direction of the analysis strip 100c.

Once a sample is introduced into the loading pad 2, a flow of the sample starts due to a capillary phenomenon. Accordingly, the sample may pass through the membrane 4 and end by being absorbed by the absorption pad 5.

The loading pad 2, the membrane 4, and the absorption pad 5 may include a material having porosity by which the sample spreads due to a capillary phenomenon, and the loading pad 2 may filter insoluble particles and impurities in the sample. A proximal end of the membrane 4 may contact a distal end the loading pad 2, and the distal end of the membrane 4 may contact a proximal end of the absorption pad 5.

The conjugating region 6, the test region 7, and the control region 8 may be arranged on the membrane 4. The conjugating region 6, the test region 7, and the control region 8 may be arranged in a linear shape so as to be spaced apart from each other. That is, the conjugating body, the first captor, and the second captor may be disposed on one membrane 4. In addition, the reference region 9 may be disposed on the loading pad 2.

Referring to FIG. 10, an analysis strip 100d may include the loading pad 2, the membrane 4, and the absorption pad 5 sequentially arranged in a lengthwise direction thereof, wherein the conjugating region 6 and the reference region 9 may be disposed on the loading pad 2.

Referring to FIGS. 9 and 10, the conjugating region 6 and the reference region 9 of the analysis strip 100c of FIG. 9 are disposed on the membrane 4 and the loading pad 2, respectively, whereas the conjugating region 6 and the reference region 9 of the analysis strip 100d of FIG. 10 are disposed on the loading pad 2. The conjugating region 6 may be disposed on any one of the loading pad 2, the conjugating pad 3, and the membrane 4 according to a sample or a target material.

Referring to FIG. 11, an analysis strip 100e may include the membrane 4 and the absorption pad 5 sequentially arranged in a lengthwise direction thereof, wherein the conjugating region 6, the test region 7, the control region 8, and the reference region 9 may be disposed on the membrane 4.

Referring to FIGS. 10 and 11, the analysis strip 100d of FIG. 10 includes the separate loading pad 2, whereas the analysis strip 100e of FIG. 11 does not include the loading pad 2. If it is not necessary to preprocess and modify or change characteristics of a sample for a pad, the loading pad 2 may be omitted. That is, a sample may be introduced into a partial region of the membrane 4. As described above, an analysis strip may include two or more pads. Types and the number of pads may be variously changed according to an introduced sample and a target material.

The analysis strip 100 shown in FIGS. 1 to 3 includes two auxiliary strips spaced apart from each other but is not limited thereto. The number of auxiliary strips may be one or more and may vary according to a precision of a quantitative analysis. In addition, when a plurality of target materials are desired to be detected, a plurality of complexes and reference materials may be prepared.

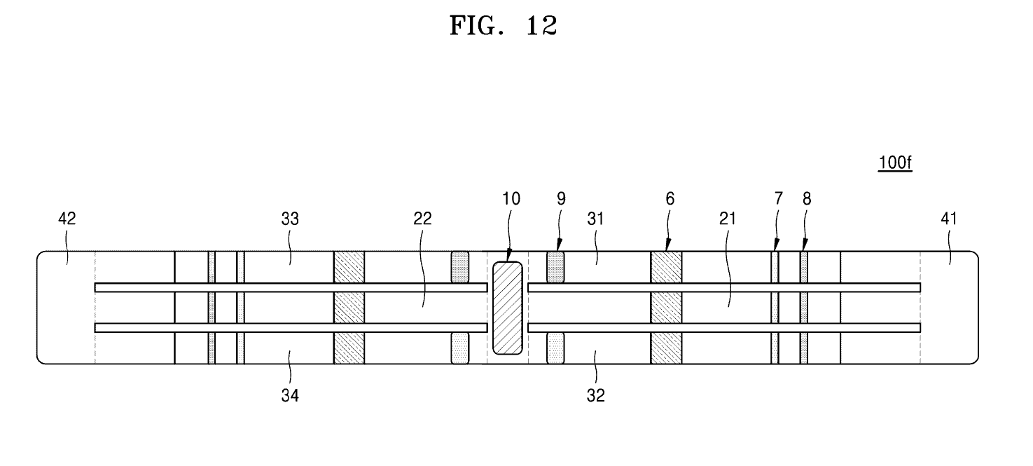

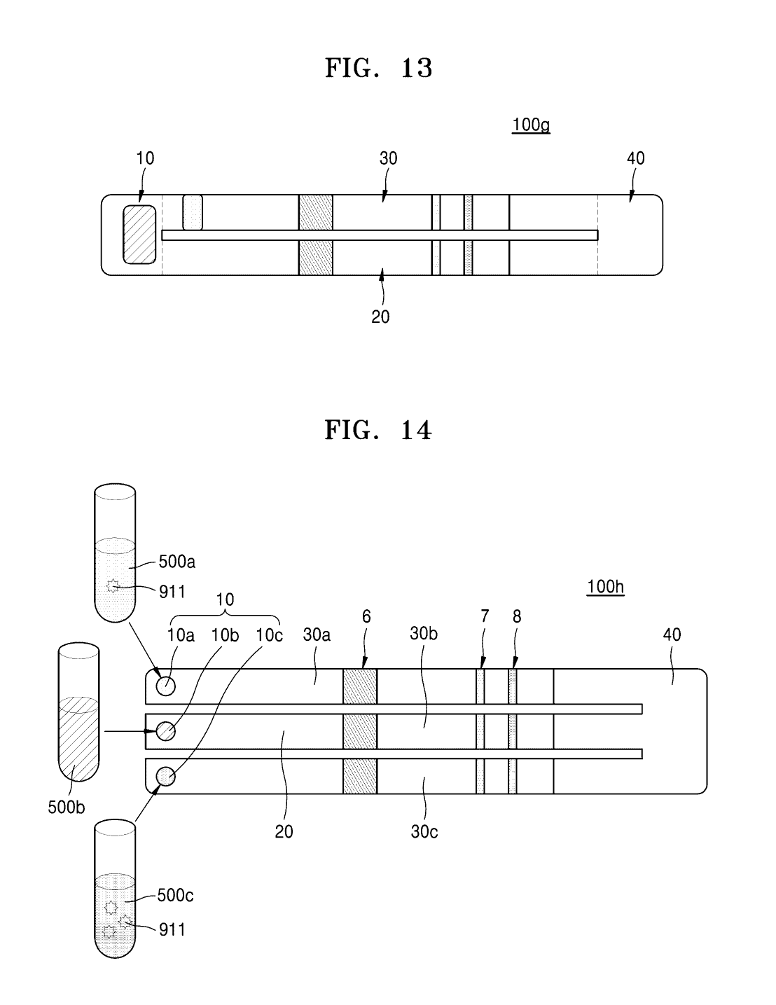

FIGS. 12 and 13 illustrate top views of analysis strips according to various embodiments of the present disclosure.

Referring to FIG. 12, the analysis strip 100f may include a single introduction part 10 through which a sample including a target material is introduced, first and second target strips 21 and 22 in which a first sample of the sample loaded from the introduction part 10 flows due to a capillary phenomenon and from which the target material in the sample is detected, first to fourth auxiliary strips 31, 32, 33, and 34 that are spaced apart from the first and second target strips 21 and 22, in which a reference material is impregnated, in which a second sample of the sample loaded from the introduction part 10 flows due to a capillary phenomenon, and from which the reference material and the target material are detected, and first and second absorption parts 41 and 42 that absorb the target material and the remaining sample which has passed through the four auxiliary strips 31, 32, 33, and 34. The sample introduced into the introduction part 10 may be divided, pass through the first and second target strips 21 and 22 and the first to fourth auxiliary strips 31, 32, 33, and 34, and then be absorbed by the first and second absorption parts 41 and 42.

The first and second target strips 21 and 22 may detect the same target material. For example, the same conjugating body, the same first captor, and the same second captor may be respectively disposed in the conjugating region 6, the test region 7, and the control region 8 of the first and second target strips 21 and 22 and the first to fourth auxiliary strips 31, 32, 33, and 34. In addition, a reference material having different densities may be impregnated in at least two of the first to fourth auxiliary strips 31, 32, 33, and 34. The number of auxiliary strips or reference regions having different densities corresponds to a precision of a quantitative analysis on the target material.

Alternatively, different target materials may be detected from the first and second target strips 21 and 22. One sample may include a plurality of target materials. For example, when the sample is blood, diabetes may be diagnosed by evaluating glucose and osteoporosis may be diagnosed by evaluating C-terminal telopeptide (CTx). The analysis strip 100f according to an embodiment may simultaneously detect a plurality of target materials by using one sample.

For example, a conjugating body, a first captor, and a second captor for detecting a first target material may be respectively disposed in the conjugating region 6, the test region 7, and the control region 8 of the first target strip 21 and the first and second auxiliary strips 31 and 32. In addition, a conjugating body, a first captor, and a second captor for detecting a second target material that is different from the first target material may be respectively disposed in the conjugating region 6, the test region 7, and the control region 8 of the second target strip 22 and the third and fourth auxiliary strips 33 and 34.

In addition, a reference material (e.g., the first target material) having different densities may be disposed in the reference region 9 of the first and second auxiliary strips 31 and 32, and a reference material (e.g., the second target material) having different densities may be disposed in the reference region 9 of the third and fourth auxiliary strips 33 and 34. Accordingly, a qualitative analysis and a quantitative analysis on the first and second target materials may be performed with one sample.

Although FIG. 12 shows two target strips and four auxiliary strips, the present embodiment is not limited thereto. An analysis strip may include one or more target strips and three or more auxiliary strips. The numbers of target strips and auxiliary strips may vary according to types of target materials or a precision of a quantitative analysis.

Referring to FIG. 13, the analysis strip 100g may include one introduction part 10 through which a sample including a target material is introduced from the outside, one target strip 20 in which a first sample of the sample loaded from the introduction part 10 flows due to a capillary phenomenon and from which the target material in the sample is detected, one auxiliary strip 30 which is spaced apart from the target strip 20, in which a reference material is impregnated, in which a second sample of the sample loaded from the introduction part 10 flows due to a capillary phenomenon, and from which the reference material and the target material are detected, and one absorption part 40 which absorbs the remaining sample which has passed through the auxiliary strip 30. The sample introduced into the introduction part 10 may be divided, pass through the target strip 20 and the auxiliary strip 30, and then be absorbed by the absorption part 40.

When one auxiliary strip 30 is used, a precision of a quantitative analysis of the target material may be lower than a case where a plurality of auxiliary strips 30 are used. Accordingly, it may be necessary to determine whether a content of a target material is within a range of certain values, which may be determined using only one auxiliary strip 30.

Although it has been described that a reference materials is impregnated in an auxiliary strip, the various embodiments described above are not limited thereto. A reference material may be added to a sample introduced into the introduction part 10 without impregnating the reference material in an auxiliary strip.

FIG. 14 illustrates a diagram for describing a method of using an analysis strip according to an embodiment of the present disclosure.

Referring to FIG. 14, an analysis strip 100h may include first to third strips 30a, 30b, and 30c. Proximal ends of the first to third strips 30a, 30b, and 30c may be connected to first to third introduction parts 10a, 10b, and 10c, respectively, and the distal ends of the first to third strips 30a, 30b, and 30c may be connected to the absorption part 40. The conjugating region 6, the test region 7, and the control region 8 are disposed at corresponding locations of each of the first to third strips 30a, 30b, and 30c.

The introduction part 10 may include the first to third introduction parts 10a, 10b, and 10c respectively connected to the first to third strips 30a, 30b, and 30c and spaced apart from each other. A user collects a sample and then divides the sample into first to third samples 500a, 500b, and 500c. A reference material 911 of a first density may be added to the first sample 500a, and the reference material 911 of a second density may be added to the third sample 500c.

The first and third samples 500a and 500c to which the reference material 911 is added may be applied to the first and third introduction parts 10a and 10c, respectively, and the second sample 500b may be applied to the second introduction part 10b. Then, a similar result as that of the analysis strip 100 shown in FIG. 1 may be obtained.

Although it has been described that a conjugating body is disposed in the first to third strips 30a, 30b, and 30c, the present embodiment is not limited thereto. The conjugating body may be added to the first to third samples 500a, 500b, and 500c before the sample is loaded on the analysis strip 100h.

Although rectangular shaped strips for analysis have been described, the embodiments described above are not limited thereto. An analysis strip may have a different shape according to various embodiments of the present disclosure.

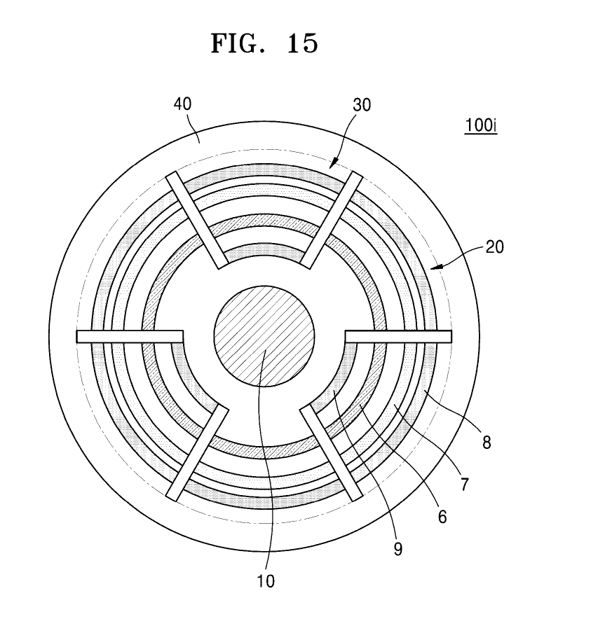

FIG. 15 illustrates an analysis strip according to an embodiment of the present disclosure.

Referring to FIG. 15, the analysis strip 100i may be circular-shaped. In the analysis strip 100i, one or more target strips 20 and one or more auxiliary strips 30 may be radially arranged around the introduction part 10. In addition, the absorption part 40 may be disposed at a circumferential edge of the analysis strip 100i so as to enclose the target strips 20 and the auxiliary strips 30.

When a plurality of target strips 20 are used, the same target material may be detected, or different target materials may be detected. A reference material that is the same as a target material to be detected from a neighboring target strip 20 may be impregnated in the auxiliary strip 30. In addition to the circular-shaped analysis strip 100i, strips for analysis of various shapes may be implemented.

The analysis strip 100 described above may include a cartridge for providing a housing.

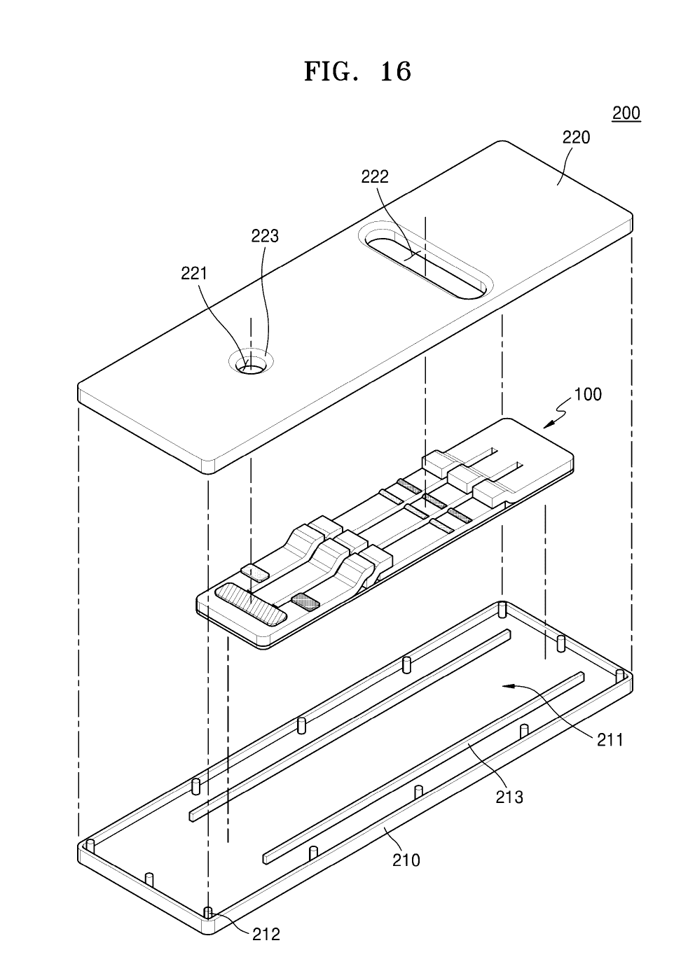

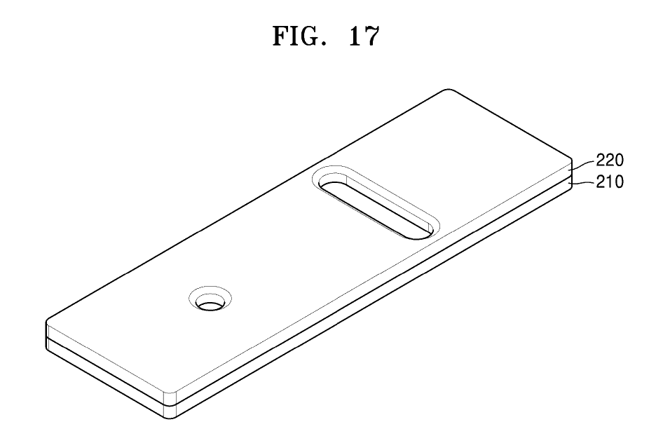

FIG. 16 illustrates an exploded perspective view of a cartridge including the analysis strip, according to an embodiment of the present disclosure, and FIG. 17 illustrates an outer appearance perspective view of the cartridge of FIG. 16 according to an embodiment of the present disclosure.

Referring to FIGS. 16 and 17, a cartridge 200 may enclose an analysis strip that is capable of quantitatively analyzing the target material by using immunochromatography. The cartridge 200 may include the analysis strip 100, a first housing 210 configured to accommodate the analysis strip 100 therein, and a second housing 220 configured to cover an upper end of the first housing 210.

The first housing 210 may include a strip accommodation part 211 configured to accommodate the analysis strip 100 and a first coupling part 212 coupled to the second housing 220. The strip accommodation part 211 may include a plurality of guides 213 protruding from the first housing 210. The guides 213 may allow the analysis strip 100 to be located at a pre-determined location and may prevent movement of the analysis strip 100. The first coupling part 212 may be coupled to a second coupling part (not shown) disposed in the second housing 220 such that the second housing 220 is fitted to the first housing 210 and is closed. The first coupling part 212 and the second coupling part may be sealed to be waterproof and aerosol proof.

The second housing 220 may include an introduction hole 221 through which a sample may be introduced from the outside and a transparent or translucent observation window 222 through which a reaction result which has occurred in the analysis strip 100 may be measured or confirmed.

The introduction hole 221 may be located at a location corresponding to the introduction part 10 when the second housing 220 is coupled to the first housing 210. The introduction hole 221 may be formed in a circular shape as shown in FIG. 16 but is not limited thereto. The introduction hole 221 may be formed in a polygonal shape. A user may drop a sample to be analyzed on the introduction hole 221 by using a tool such as a pipet, but the present embodiment is not limited thereto. The user may dip the introduction hole 221 into a place where a sample flows so that the sample flows into the cartridge 200.

The second housing 220 may further include, around the introduction hole 221, an introduction guide part 223 that is inclined in a direction of the introduction hole 221. The introduction guide part 223 guides a sample dropping in the surroundings of the introduction hole 221 to flow into the introduction hole 221. In detail, when the user does not correctly drop a sample into the introduction hole 221 such that a portion of the sample drops in the surroundings of the introduction hole 221, the sample dropping in the surroundings of the introduction hole 221 flows into the introduction hole 221 by the inclination of the introduction guide part 223. In addition, since the introduction guide part 223 protrudes towards the inside of the second housing 220, when the second housing 220 is coupled to the first housing 210, the introduction guide part 223 may function to fix the position of the analysis strip 100.

The observation window 222 may be disposed in the second housing 220 at a location to view the test region 7 and the control region 8. In this case, the user may confirm degrees of color development of the test region 7 and the control region 8 through the observation window 222.

Alternatively, the observation window 222 may be disposed at a location corresponding to the test region 7, the control region 8, and the absorption part 40. In this case, the user may confirm degrees of color development of the test region 7, the control region 8, and the absorption part 40 through the observation window 222. Although the observation window 222 is shown as an oval shape, the observation window 222 is not limited thereto and may be formed in a polygonal shape.

The cartridge 200 according to an embodiment may further include an observation window cover (not shown). The observation window cover may protect the cartridge 200. For example, the observation window cover may prevent damage during transportation, prevent a sample from being introduced into the strip due to a mistake of a user, prevent an internal water vapor condensation phenomenon occurring due to a temperature difference from the outside, and protect the strip from scratches or contamination.

The observation window cover is attached to the second housing 220 and uses an open/close method, and when the cartridge 200 is not used, the observation window cover may cover the observation window 222. The observation window cover may have a semiautomatic structure in which the observation window cover is opened in a sliding mechanism when the cartridge 200 is inserted into a reader to read a reaction result after a flowing analysis, or have a structure manually opened or closed by a user.

The first and second housings 210 and 220 may be manufactured using chemically stable synthetic resins and a combination thereof. For example, the first and second housings 210 and 220 may be manufactured by a well-known forming method by using various thermosetting and thermoplastic plastics, such as polyethylene, polypropylene, polystyrene, polyethylene terephthalate, polyamide, polyester, polyvinyl chloride, polyurethane, polycarbonate, polyvinylidene chloride, tetrafluoromethylene, and polyetherimide, and a combination thereof. However, the first and second housings 210 and 220 are not limited thereto and may use any material suitable for the purpose of the cartridge 200.

The shapes of the first and second housings 210 and 220 may correspond to the shape of the analysis strip 100. For example, when the analysis strip 100 is polygonal shaped, the first and second housings 210 and 220 may also be polygonal shaped, and when the analysis strip 100 is circular shaped, the first and second housings 210 and 220 may also be circular shaped. However, the shapes of the first and second housings 210 and 220 are not limited thereto. The shapes of the first and second housings 210 and 220 may be irrelevant to the shape of the analysis strip 100.

The analysis strip 100 may be disposed between the first and second housings 210 and 220 or may be one component of another device. For example, the strip for analysis 100 may be attached to a location contactable with a human body in a wearable device.

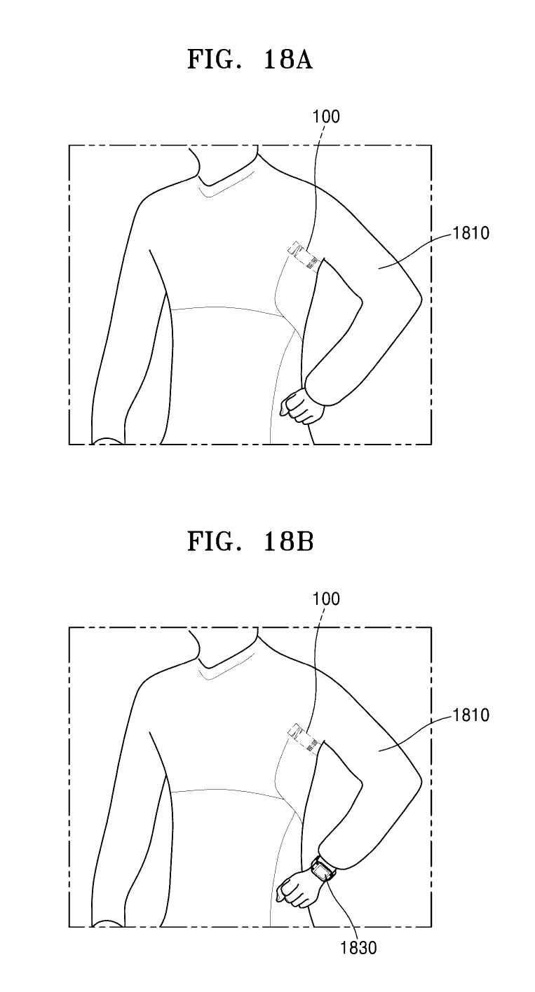

FIGS. 18A and 18B illustrate an analysis strip attached to wearable items according to an embodiment of the present disclosure.

Referring to FIG. 18A, when a sample is a body fluid, the analysis strip 100 may be attached in a region of a wearable item 1810, which corresponds to a region from which the body fluid is fluently secreted, e.g., an armpit. However, the attachment region is not limited thereto. The analysis strip 100 may also be disposed in another region of the wearable item 1810. For example, the analysis strip 100 may be attached in a region of the wearable item 1810 that corresponds to a chest. In addition, a plurality of strips for analysis 100 may be attached to the wearable item 1810.

In addition, the wearable item 1810 may include at least one of an analysis reader (not shown) configured to read a degree of color development of the analysis strip 100 and an analysis apparatus (not shown). In this case, the analysis reader and the analysis apparatus may acquire information related to a target material by using a result detected by the analysis strip 100 and output the information as a sound, vibrations, or the like. FIG. 18A shows a light-emitting unit configured to output the target information as a color.

Referring to FIG. 18B, the analysis apparatus may be separate from the wearable item 1810. For example, the analysis apparatus may include a wearable device 1830 such as a smartwatch, a separate device connected to a smartphone, a smartphone, or the like. The user may separate the analysis strip 100 from the wearable item 1810 and insert the analysis strip 100 into the wearable device 1830. In this case, the wearable device 1830 may acquire information related to a target material from the analysis strip 100. The wearable device 1830 may output the acquired information. Alternatively, the analysis reader may be included in the wearable item 1810 and optical information acquired by the analysis reader may be transmitted to the wearable device 1830. The analysis reader may transmit the optical information to the wearable device 1830 through wireless communication. In this case, the wearable device 1830 may acquire information related to a target material from the optical information and provide a result thereof.

Although the wearable device 1830 is shown as the analysis apparatus, the analysis apparatus is not limited thereto. The analysis apparatus may be a portable device of a user or various types of electronic devices configured to acquire the information related to a target material from optical information.

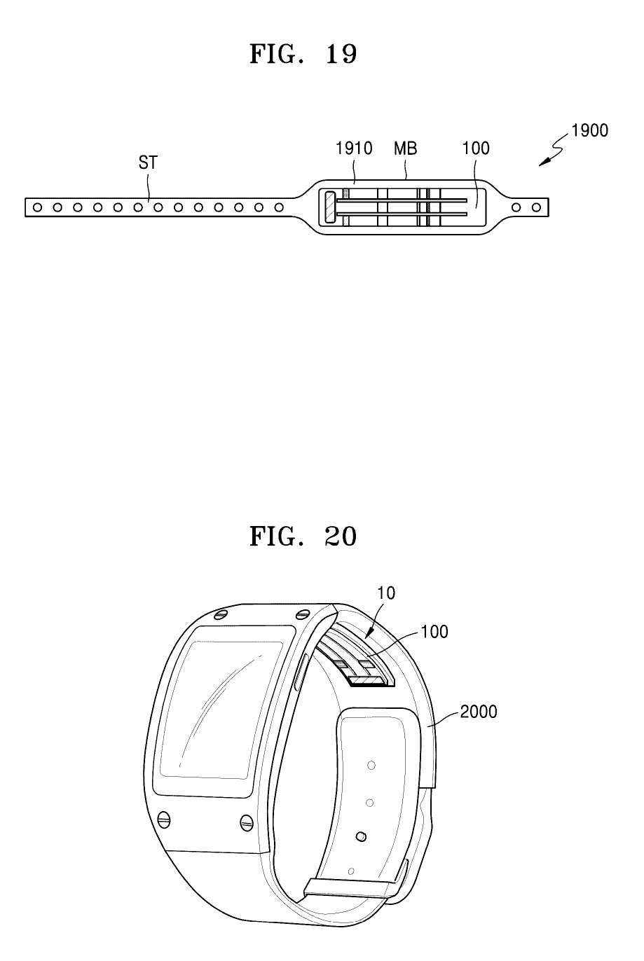

FIG. 19 illustrates a diagram for describing a band-type cartridge according to an embodiment of the present disclosure.

Referring to FIG. 19, a band-type cartridge 1900 may include a main body part (MB) and a strap (ST). The strap ST is provided at both sides of the main body part MB so as to be wearable around a wrist or the like of a user by being connected to the main body part MB. The main body part MB has, on a rear surface thereof, an accommodation part 1910 configured to accommodate the analysis strip 100, and the analysis strip 100 may be attached to or detached from the accommodation part 1910 of the band-type cartridge 1900. A user may wear the band-type cartridge 1900 such that the analysis strip 100 contacts the skin.

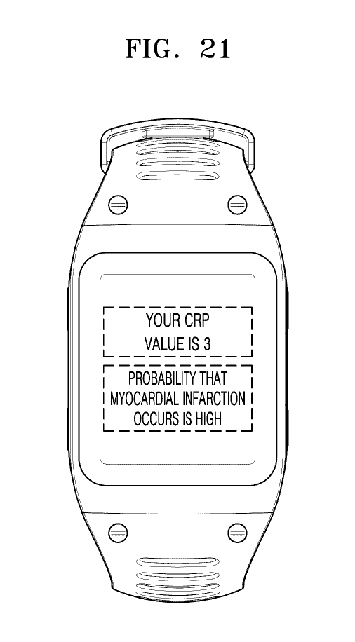

FIG. 20 illustrates a diagram for describing the analysis strip which is attachable to and detachable from a smartwatch according to an embodiment of the present disclosure, and FIG. 21 illustrates a reference diagram for describing a method by which the smartwatch of FIG. 20 displays an analysis result, according to an embodiment of the present disclosure.

Referring to FIG. 20, when a wearable device is configured as a smartwatch 2000, the analysis strip 100 may be attached to a rear surface of the smartwatch 2000. In this case, when a user wears the smartwatch 2000, the analysis strip 100 may contact the skin of the user, and a body fluid secreted from the skin of the user may flow into the introduction part 10 of the analysis strip 100. In addition, the smartwatch 2000 may include a reader (not shown) configured to read a degree of color development of the analysis strip 100 and an analysis apparatus (not shown). Referring to FIG. 20, the smartwatch 2000 may output an analysis result.

The analysis strip 100 according to an embodiment may be a disposable strip which cannot be reused after an analysis. However, the analysis strip 100 is not limited thereto. If a result according to an immune reaction of the analysis strip 100 is an oxidation reaction, the analysis strip 100 may be initialized through a reduction reaction, e.g., exposure to air or addition of a solution. In this case, the analysis strip 100 may be continuously used without being replaced by a new analysis strip 100 each time that an analysis result is outputted.

Whether a sample includes a target material or an inclusion degree of the target material may be determined based on a degree of color development of the test region included in the analysis strip 100, which has been described above. When the degree of color development is confirmed with the naked eyes, a user may directly analyze an analysis result based on the degree of color development. However, an analysis reader and an analysis apparatus to be described below may more correctly perform a quantitative analysis than the naked eyes.

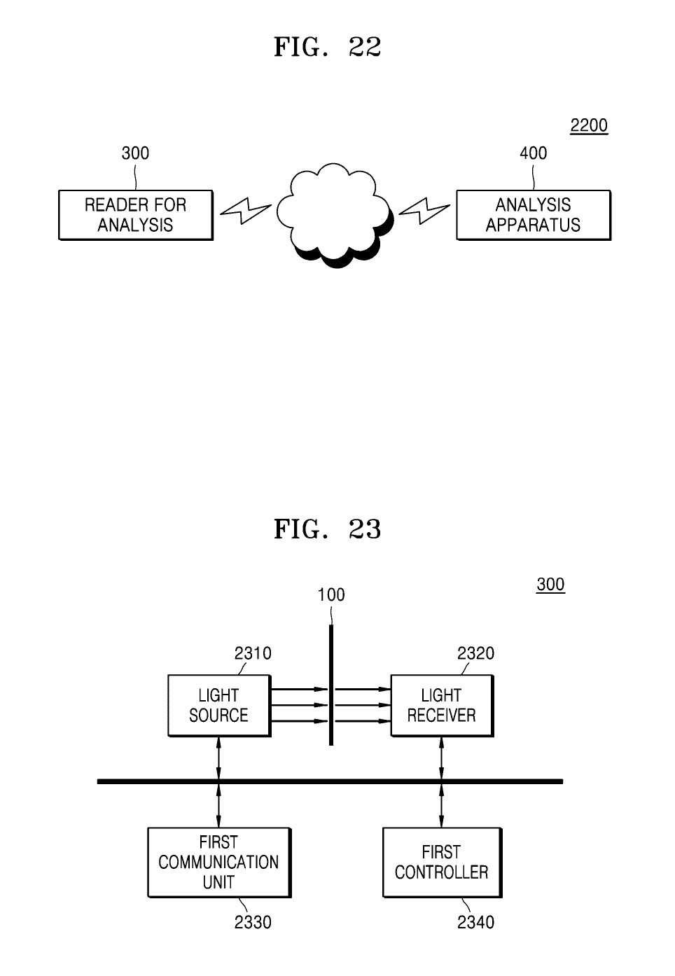

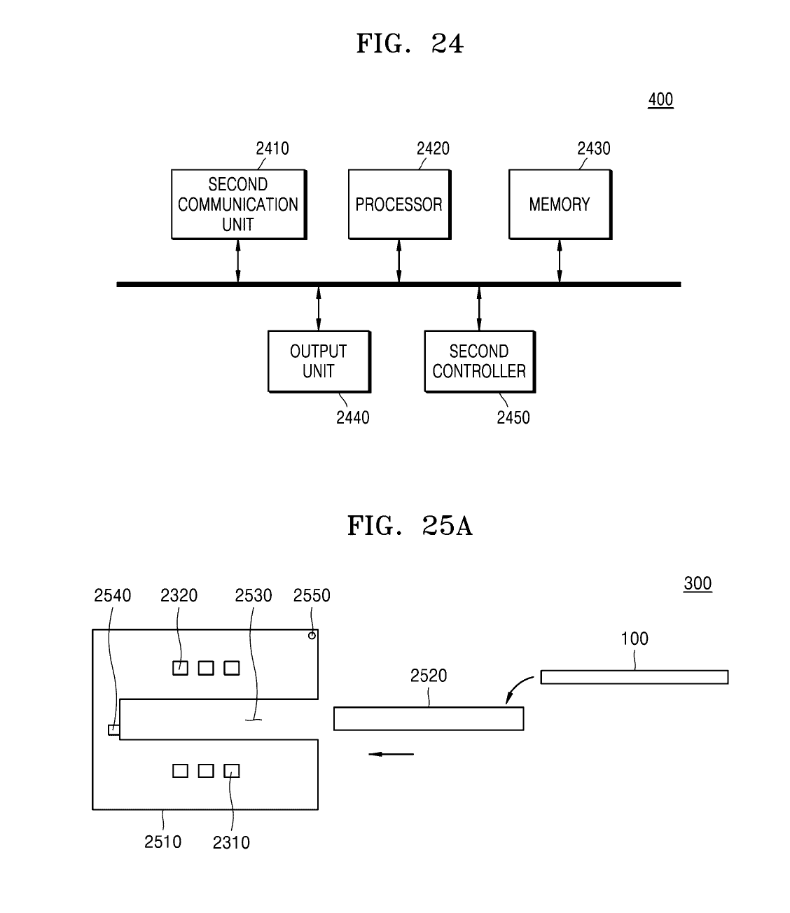

FIG. 22 illustrates a block diagram of an analysis system according to an embodiment of the present disclosure, FIG. 23 illustrates a block diagram of an analysis reader in the analysis system of FIG. 22 according to an embodiment of the present disclosure, and FIG. 24 illustrates a block diagram of an analysis apparatus in the analysis system of FIG. 22 according to an embodiment of the present disclosure.

Referring to FIG. 22, an analysis system 2200 may include an analysis reader 300 and is configured to detect a degree of color development of a test region of an analysis strip and an analysis apparatus 400 is configured to analyze a target material based on the degree of color development of the test region, which is received from the analysis reader 300. The analysis reader 300 and the analysis apparatus 400 may be implemented as one apparatus or implemented as independent separate apparatuses.

When the analysis reader 300 and the analysis apparatus 400 are independent separate apparatuses, the analysis reader 300 and the analysis apparatus 400 may communicate in a wired or wireless manner. When the analysis reader 300 and the analysis apparatus 400 communicates in a wired or wireless manner, each of the analysis reader 300 and the analysis apparatus 400 may include a communication unit.

Hereinafter, for convenience of description, the analysis reader 300 and the analysis apparatus 400 are described as independent separate apparatuses. However, the analysis reader 300 and the analysis apparatus 400 are not limited thereto. For example, the analysis reader 300 and the analysis apparatus 400 may be implemented as one apparatus. When the analysis reader 300 and the analysis apparatus 400 are implemented as one apparatus, the communication unit for communication between the analysis reader 300 and the analysis apparatus 400 may not be necessary.

Referring to FIG. 23, the analysis reader 300 according to an embodiment may include a light source 2310 configured to emit light onto the analysis strip 100, a light-receiver 2320 configured to receive light from the analysis strip 100, a first communication unit 2330 configured to transmit information related to the light received in the analysis apparatus 400, and a first controller 2340 configured to control a general operation of the analysis reader 300.

The analysis reader 300 according to an embodiment may acquire information related to a target material by using laser-induced fluorescence detection. For example, the light source 2310 may induce emission of the color development particle 112 in the test region 7 by emitting light of a wavelength band corresponding to the color development particle 112. For example, the light source 2310 may include a light-emitting diode (LED), an infrared (IR) light source, an ultraviolet (UV) light source, a nano-light source, or the like. The induced light may be detected by the light-receiver 2320. A filter or the like configured to selectively detect the emitted light may be disposed between the light-receiver 2320 and the analysis strip 100, and a filter, e.g., an object lens, configured to filter incident light may also be disposed between the light source 2310 and the analysis strip 100.

When the target material is detected by laser-induced fluorescence detection, the color development particle 112 may include a fluorescent material of which an absorption wavelength is different from a discharge wavelength. For example, a difference between the absorption wavelength and the discharge wavelength may be about 20 nm or more but is not limited thereto. The fluorescent material may include a fluorescent particle, a quantum dot, a lanthanide chelate (e.g., samarium (Sm), europium (Eu), or terbium (Tb)), fluorescence (e.g., fluorescein Isothiocyanate (FITC), Rhodamine Green, thiacarbocyanine, Cy2, Cy3, Cy5, Cy5.5, Alexa 488, Alexa 546, Alexa 594 or Alexa 647), or the like. To detect deoxyribonucleic acid (DNA), Cy3, Cy5, or the like may be used as the fluorescent material. The intensity of fluorescent light may be generally proportional to the intensity of excitation light if the intensity of excitation light is not excessively high.

Alternatively, the analysis reader 300 may acquire information related to a target material by using a light emitting diode (LED). For example, the LED light may be diffused LED light. When the light is emitted from a LED, the light-receiver 2320 may include a general image sensor such as a complementary metal oxide semiconductor (CMOS) or a charge coupled device (CCD).

Alternatively, the analysis reader 300 according to an embodiment may not separately include the light source 2310. When the color development particle 112 is recognizable with the naked eyes or is a pigment recognizable in a visible light band, the analysis reader 300 may include the light-receiver 2320 including an image sensor without including the light source 2310.

The first communication unit 2330 may transmit information related to received light (hereinafter, referred to as “optical information”) to the analysis apparatus 400. The optical information may include information related to the intensity of light, a wavelength band of the light, and the like.

The first controller 2340 may control a general operation of the analysis reader 300. If the analysis strip 100 is inserted into the analysis reader 300, the first controller 2340 may control the light source 2310, the light-receiver 2320, and the first communication unit 2330 to be activated. For example, the first controller 2340 may control the light source 2310 to emit light onto the strip, control the light-receiver 2320 to receive light, and control the first communication unit 2330 to transmit optical information to the analysis apparatus 400.

Referring to FIG. 24, an analysis apparatus 400 may include a second communication unit 2410 configured to communicate with the analysis reader 300, a processor 2420 configured to acquire information related to a target material (hereinafter, referred to as “target material information”) by using optical information, a memory 2430 in which data for analyzing the target material information and the like are stored, an output unit 2440 configured to output the target material information, and a second controller 2450 configured to control a general operation of the analysis apparatus 400.

The second communication unit 2410 receives optical information from the analysis reader 300. The second communication unit 2410 may communicate with the analysis reader 300 in a wired or wireless manner.

The processor 2420 may acquire target material information corresponding to the optical information. The analysis reader 300 acquires optical information including the intensity, a density, and the like of light incident from the test region 7 and transmits the optical information to the analysis apparatus 400. The processor 2420 may acquire target material information corresponding to the optical information received from the analysis reader 300 by using a lookup table in which optical information matches target material information.

When the target material information is acquired, the processor 2420 may acquire the target material information by using an amount of a sample introduced into the analysis strip 100, user information (e.g., age, sex, and medical record information), a geological location where a diagnosis was performed, a time the diagnosis was performed, information related to the strip (e.g., a manufacturer and a manufacturing date of the strip), and the like.

In addition, the processor 2420 may acquire target material information by using optical information detected from the target strip 20 (hereinafter, referred to as “first optical information”) and optical information detected from the auxiliary strip 30 (hereinafter, referred to as “second optical information”). For example, when the analysis strip 100 includes one target strip 20 and two auxiliary strips 30 having different densities of a reference material, the processor 2420 may acquire target material information by using one piece of first optical information acquired from the one target strip 20 and two pieces of second optical information acquired from the two auxiliary strips 30.

The memory 2430 may store data generated during an operation of the analysis apparatus 400. The memory 2430 may store a lookup table in which optical information matches density information of target materials.

The memory 2430 may include a hard disk drive (HDD), read only memory (ROM), random access memory (RAM), flash memory, and a memory card.

The output unit 2440 may output target material information. The output unit 2440 may include at least one of a display configured to display the target material information as an image or a text and a speaker configured to output the target material information as an audible frequency. In addition, the output unit 2440 may further include a vibrator configured to output, as vibrations, an alarm, a light-emitting unit configured to output light, and the like. The target material information may be transmitted to an external device through the second communication unit 2410.

The second controller 2450 may control a general operation of the analysis apparatus 400 to acquire target material information. The second controller 2450 may determine whether a contained percentage of the target material 511 is normal or abnormal based on the acquired target material information and provide the determination result to a user through the display. In addition, the second controller 2450 may control the analysis reader 300. For example, when the analysis apparatus 400 and the analysis reader 300 are in a communicable state and the analysis strip 100 is inserted into the analysis reader 300, the second controller 2450 may control the analysis reader 300 so that the light source 2310 of the analysis reader 300 emits light onto the strip and the light-receiver 2320 receives light outputted from the strip.

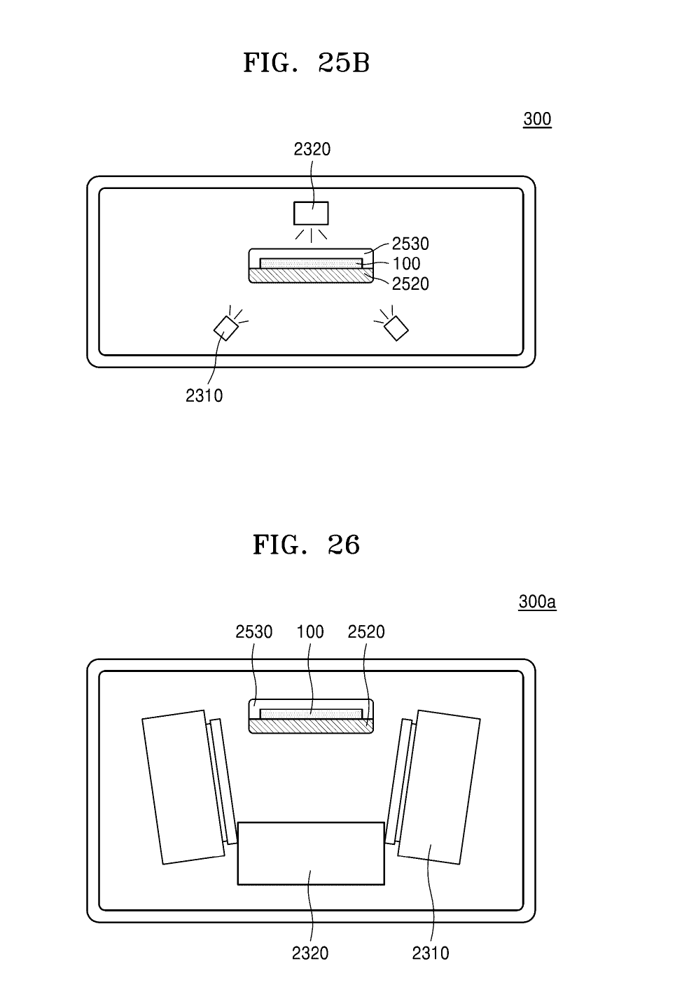

FIG. 25A illustrates a cross-sectional view of an analysis reader according to an embodiment of the present disclosure, and FIG. 25B illustrates a cross-sectional view of the analysis reader of FIG. 25A in another point of view according to an embodiment of the present disclosure.

Referring to FIGS. 25A and 25B, in the analysis reader 300, the light source 2310 and the light-receiver 2320 may be disposed in one housing 2510. Although FIG. 25A shows a plurality of light sources 2310 and a plurality of light-receivers 2320, this is only for convenience of description, and the present embodiment is not limited thereto. The number of light sources 2310 and the number of light-receivers 2320 may be modified. The light source 2310 and the light-receiver 2320 have been described above, and thus a detailed description thereof is omitted.

The housing 2510 may fix the analysis strip 100 and block ambient light. Accordingly, the light-receiver 2320 may receive light emitted from the light source 2310 included in the analysis reader 300 without receiving external light or sunlight.

The analysis reader 300 may further include a strip tray 2520 configured to support the analysis strip 100. A size and a shape of the strip tray 2520 may be formed enough to accommodate the analysis strip 100 therein. For example, the size and the shape of the strip tray 2520 may be a slightly greater than a size and a shape of the analysis strip 100. The strip tray 2520 may be reusable or disposable.

The analysis reader 300 may further include an accommodation part 2530 configured to accommodate the strip tray 2520 inside the housing 2510. The strip tray 2520 may be inserted into the accommodation part 2530 in the housing 2510 of the analysis reader 300.

The analysis reader 300 according to an embodiment may further include a sensor 2540 configured to detect that the strip tray 2520 is properly inserted into the housing 2510. For example, the sensor 2540 may prevent the light source 2310 from operating until the strip tray 2520 is properly inserted.

The analysis reader 300 may further include an output unit 2550 configured to output an alarm as light, the alarm indicating that the strip tray 2520 is properly inserted into the analysis reader 300.

FIGS. 25A and 25B show a transmissive reader in which the light-receiver 2320 of the analysis reader 300 receives light which has transmitted through the strip. However, the present embodiment is not limited thereto. The analysis reader 300 may be a reflective reader configured to receive light reflected from the analysis strip 100. Whether the analysis reader 300 is a reflective type or a transmissive type may be determined according to a type of a target material, a type of a sample, a type of a strip for analysis, a design purpose of a designer, and the like.

FIG. 26 illustrates a diagram of a reflective reader according to an embodiment of the present disclosure.

Referring to FIG. 26, a reflective reader 300a is illustrated. When a space is divided into two regions based on the analysis strip 100, the light source 2310 and the light-receiver 2320 may be disposed in the same region. For example, the accommodation part 2530 for the analysis strip 100 may be disposed in a lower region of the housing 2510, and the light source 2310 may be disposed at both sides of the housing 2510. In addition, the light-receiver 2320 may be disposed in an upper region of the housing 2510.

In this case, when the light sources 2310 emit light onto the analysis strip 100, the light-receiver 2320 may receive light reflected from the analysis strip 100. To increase light-receiving efficiency, a plurality of light sources 2310 may be disposed. In addition, a filter configured to filter light other than the reflected light may be further disposed at a light-receiver side. The reflective reader 300a may have a narrow thickness. In addition, although not shown, the reflective reader 300a may further include a lens array configured to control an optical path.

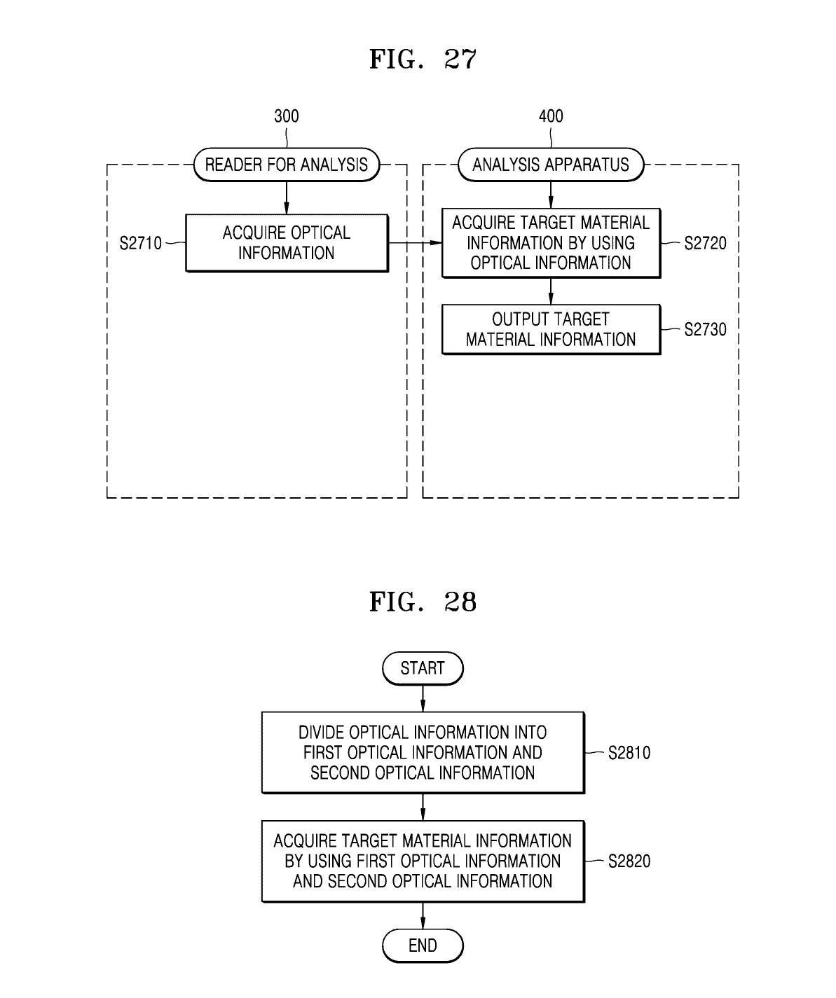

FIG. 27 is a flowchart of method for analyzing a sample according to an embodiment of the present disclosure.

Referring to FIG. 27, when the analysis strip 100 is inserted into the analysis reader 300, the analysis reader 300 acquires optical information in operation S2710. For example, the light source 2310 may emit light onto the analysis strip 100, and the light-receiver 2320 may receive light from the analysis strip 100, thereby acquiring optical information. The light-receiver 2320 may receive both light from the target trip 20 and light from the auxiliary strip 30. The analysis reader 300 may transmit the acquired optical information to the analysis apparatus 400.

The analysis apparatus 400 may acquire target material information by using the optical information in operation S2720. The processor 2420 of the analysis apparatus 400 may acquire the target material information by using at least one of optical information detected from the target strip 20 (hereinafter, referred to as “first optical information”) and optical information detected from the auxiliary strip 30 (hereinafter, referred to as “second optical information”). The processor 2420 may first determine concentration information based on the first optical information and second concentration information based on the second optical information by using a lookup table in which the optical information matches concentration information. The first concentration information may be concentration information of a target material, and the second concentration information may be concentration information of the target material and a reference material. Final target material information may be acquired by compensating for the first concentration information by using the second concentration information.

The analysis apparatus 400 may output the acquired target material information in operation S2730. The analysis apparatus 400 may display the target material information or transmit the target material information to the outside. When outputting the target material information, the analysis apparatus 400 may determine whether the target material information is normal or abnormal and display normality or abnormality, or provide a guide for living according to an analysis on the target material information.

FIG. 28 is a flowchart of a method for acquiring target material information according to an embodiment of the present disclosure.

Referring to FIG. 28, in operation S2810, the analysis apparatus 400 may divide optical information received from the analysis reader 300 into first optical information corresponding to the target strip 20 and second optical information corresponding to the auxiliary strip 30. The first optical information and the second optical information may be identified using relative locations of the target strip 20 and the auxiliary strip 30. The analysis apparatus 400 may receive the optical information from the analysis reader 300 as an image type. For example, the first auxiliary strip 31 and the second auxiliary strip 31 are spaced apart from each other by interposing the target strip 20 therebetween, the analysis apparatus 400 may determine optical information corresponding to the center as the first optical information corresponding to the target strip 20 and determine optical information corresponding to the both edges as the second optical information corresponding to the auxiliary strip 30.

In operation S2820, the analysis apparatus 400 may acquire target material information (i.e., concentration information) of the target material 511 by using the first optical information and the second optical information.

The optical information acquired from the analysis strip 100 has a correlation with concentration information of a material detected from the test region 7.

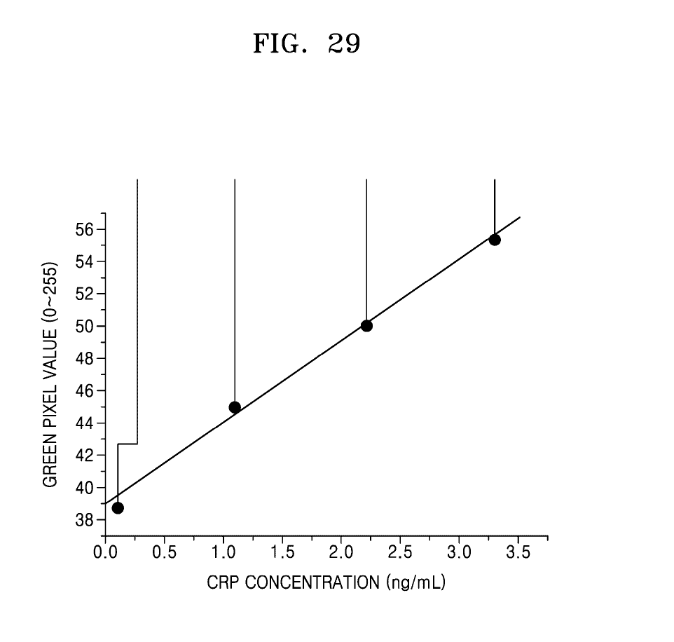

FIG. 29 illustrates a graph of a correlation between optical information and concentration information according to an embodiment of the present disclosure.

Referring to FIG. 29, for example, when a material detected from the test region 7 is CRP and light outputted from a color development particle of a conjugating body is in a green wavelength band, a correlation between CRP concentration and a green wavelength may be acquired through experiments. The correlation may be stored in the memory 2430 of the analysis apparatus 400 in a lookup table form.

Thereafter, the analysis apparatus 400 may acquire the target material information (i.e., concentration information) of the target material 511 by using the first optical information and the second optical information. For example, referring back to FIG. 1, the analysis strip 100 may include the target strip 20 and the first and second auxiliary strips 31 and 32. The reference material 911 of a first concentration c1 may be impregnated in the first auxiliary strip 31, and the reference material 911 of a second concentration c2 may be impregnated in the second auxiliary strip 32. When the reference material 911 is impregnated in the first and second auxiliary strips 31 and 32, the analysis apparatus 400 may measure a solvent of a sample introduced into the analysis strip 100 and then convert an impregnation density of the reference material 911 into concentration.

The analysis apparatus 400 may acquire concentration X of the target material 511 by using concentration information of the reference material 911, first optical information acquired from the target strip 20, and second optical information acquired from the auxiliary strip 30 as expressed by Equation 1.

X=(c2−c1)Sₓ/(S_{c2+x}−S_{c1+x}) Equation 1

In Equation 1, c1 denotes concentration of the reference material 911 impregnated in the first auxiliary strip 31, c2 denotes concentration of the reference material 911 impregnated in the second auxiliary strip 32, Sₓ denotes first optical information acquired from the target strip 20, S_{c1+x} denotes second optical information acquired from the first auxiliary strip 31, and S_{c2+x} denotes second optical information acquired from the second auxiliary strip 32.

That is, the analysis apparatus 400 may determine the target material information X of the sample based on a concentration difference (S_{c2+x}−S_{c1+x}) between the reference materials 911 contained in the auxiliary strip 30, an optical information difference (c2−c1) detected from the auxiliary strip 30, and the first optical information (Sₓ) acquired from the target strip 20.

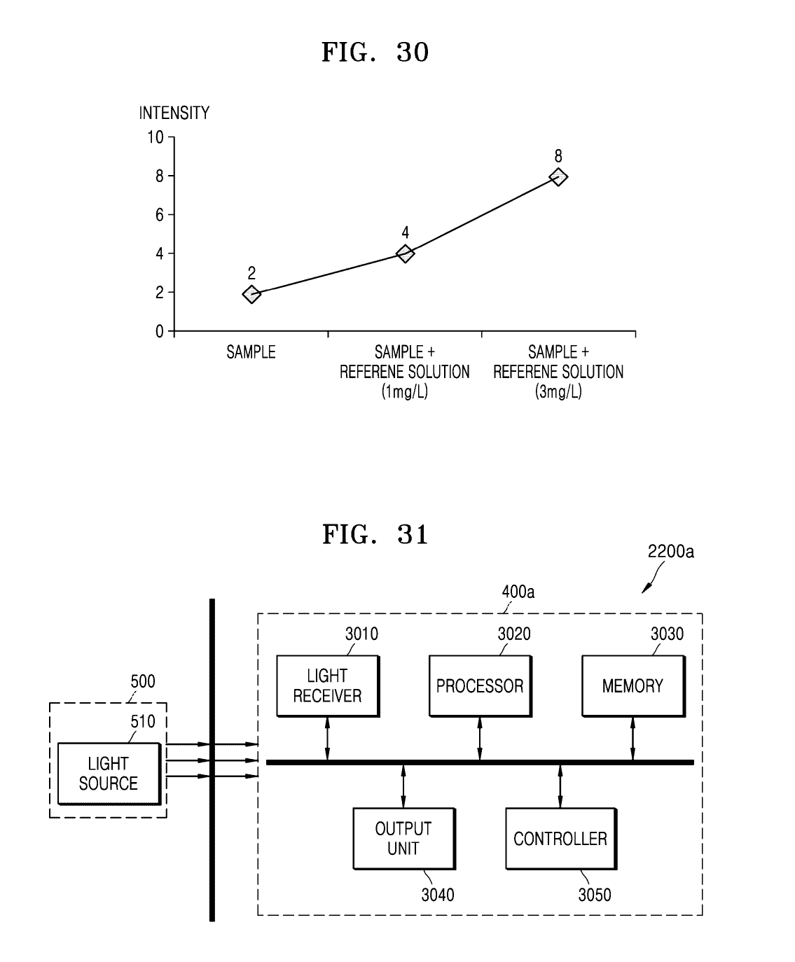

FIG. 30 illustrates a graph of target material information according to an embodiment of the present disclosure.

Referring to FIG. 30, it is presumed that concentration of glucose impregnated in the first auxiliary strip 31 is 1 mg/L and concentration of glucose impregnated in the second auxiliary strip 32 is 3 mg/L. If blood containing glucose is introduced into the analysis strip 100 according to an embodiment, one target strip 20 and two auxiliary strips 30 may detect a target material.

The analysis apparatus 400 may acquire first optical information “2” from the target strip 20, acquire second optical information “4” from the first auxiliary strip 31, and acquire second optical information “8” from the second auxiliary strip 32. In this case, the analysis apparatus 400 may acquire concentration information “1” as a result obtained by dividing a product of 2 (i.e., a concentration difference between reference materials impregnated in the auxiliary strip 30) and 2 (i.e., the first optical information acquired from the target strip 20) by 4 (i.e., a difference between the second optical information acquired from the first and second auxiliary strips 31 and 32). Then, the analysis apparatus 400 may determine concentration of the target material contained in the sample, i.e., the target material information, as “1”.

When the analysis apparatus 400 has a capturing function, the analysis apparatus 400 may perform a function of a light-receiver.

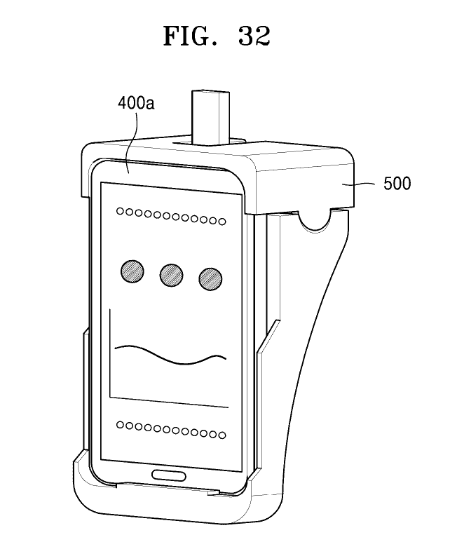

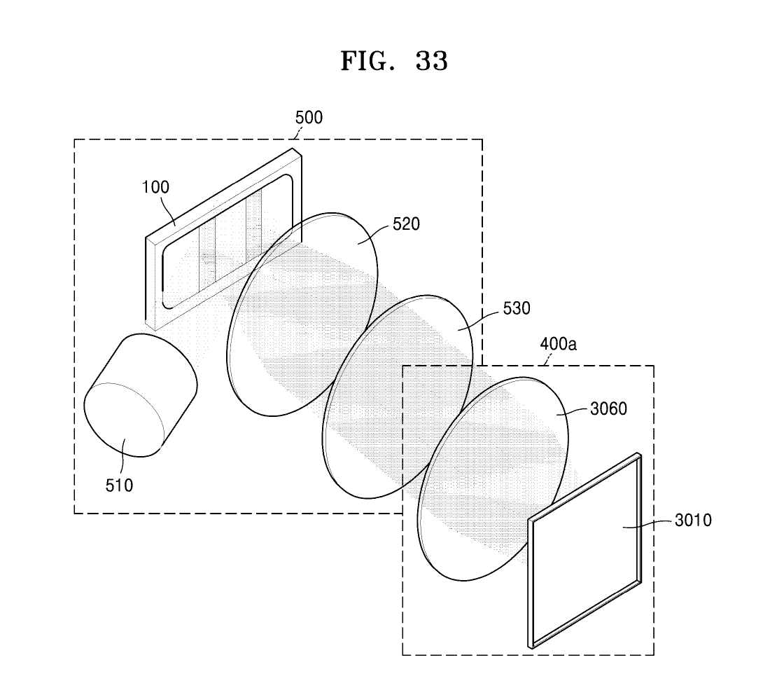

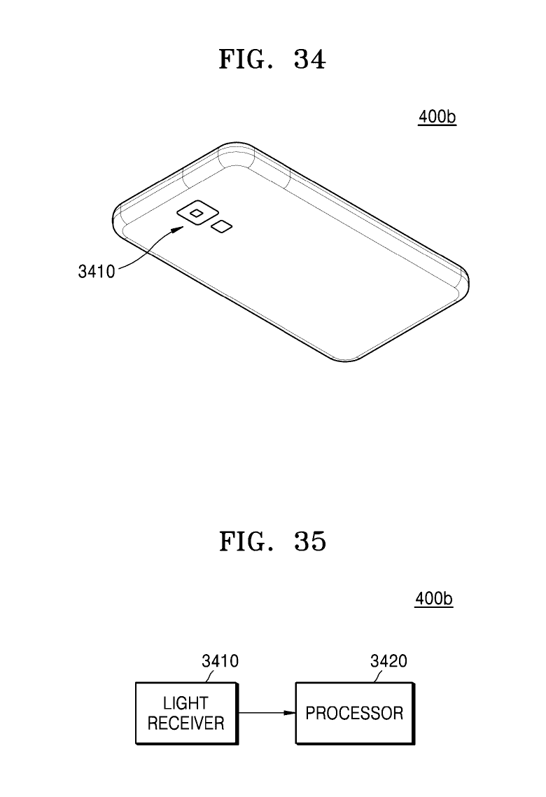

FIG. 31 illustrates a block diagram of an analysis system according to an embodiment of the present disclosure, FIG. 32 illustrates an outer appearance of the analysis system of FIG. 31 according to an embodiment of the present disclosure, and FIG. 33 illustrates an optical structure of the analysis system of FIG. 31 according to an embodiment of the present disclosure.

Referring to FIGS. 31 to 33, an analysis system 2200a may include a light source device 500 and an analysis apparatus 400a. Compared with the analysis system 2200 (see FIGS. 22 to 24) described above, the analysis apparatus 400a may include a light-receiver 3010. Since the analysis apparatus 400a directly acquires optical information, communication between the light source device 500 and the analysis apparatus 400a to acquire optical information may not have to be performed. In addition, if the analysis apparatus 400a is installed in the light source device 500, the light source device 500 may be electrically connected to a battery and a second controller 3050 of the analysis apparatus 400a through a jack (not shown) and the like. In this case, the analysis apparatus 400a may directly control a light source 510, and thus, the light source device 500 may not have to include a separate first controller.

The light source device 500 may further include a lens array 520 configured to control a light-traveling path and a filter 530 configured to cancel noise, and the analysis apparatus 400a may also further include a lens array 3060 configured to control a light-traveling path, between the light-receiver 3010 and the light source device 500. A processor 3020, a memory 3030, an output unit 3040, and a second controller 3050 of the analysis apparatus 400a are the same as described with reference to FIG. 24, and thus, a detailed description thereof is omitted.

When optical information is acquired using external light, the analysis system 2000a may include only the analysis apparatus 400a without including the light source device 500.

FIG. 34 illustrates an outer appearance of an analysis apparatus according to an embodiment of the present disclosure, and FIG. 35 illustrates a block diagram of the analysis apparatus of FIG. 35 according to an embodiment of the present disclosure.

Referring to FIGS. 34 and 35, an analysis apparatus 400b may be a general electronic device. The analysis apparatus 400b may include a light-receiver 3410 configured to acquire optical information from the analysis strip 100 and a processor 3420 configured to acquire target material information by using the optical information. The light-receiver 3410 may include a general image sensor (e.g., a CMOS or a CCD).

Referring to FIG. 34, a portable terminal may implement the analysis apparatus 400b. However, this is only illustrative, and the analysis apparatus 400b is not limited thereto. The analysis apparatus 400b may be implemented in various forms such as a device capable of performing a capturing function and executing an application for acquiring target material information (hereinafter, referred to as “diagnosis application”).

Examples of the analysis apparatus 400b are a desktop personal computer (PC), a cellular phone, a smartphone, a laptop PC, a tablet PC, an e-book terminal, a digital broadcasting terminal, a personal digital assistant (PDA), a portable multimedia player (PMP), a navigation terminal, a Moving Picture Experts Group phase 1 or phase 2 (MPEG-1 or MPEG-2) audio layer 3 (MP3) player, a digital camera, an Internet protocol television (IPTV), a digital television (DTV), and commercial electronics (CE) devices (e.g., a refrigerator and an air conditioner having a display device). Alternatively, the analysis apparatus 400b may be a wearable device wearable by a user. For example, the analysis apparatus 400b according to an embodiment may be a wrist watch, glasses, a ring, a bracelet, a necklace, or the like.

An analysis apparatus for performing a diagnosis application may acquire target material information and provide various modes for acquiring the target material information.

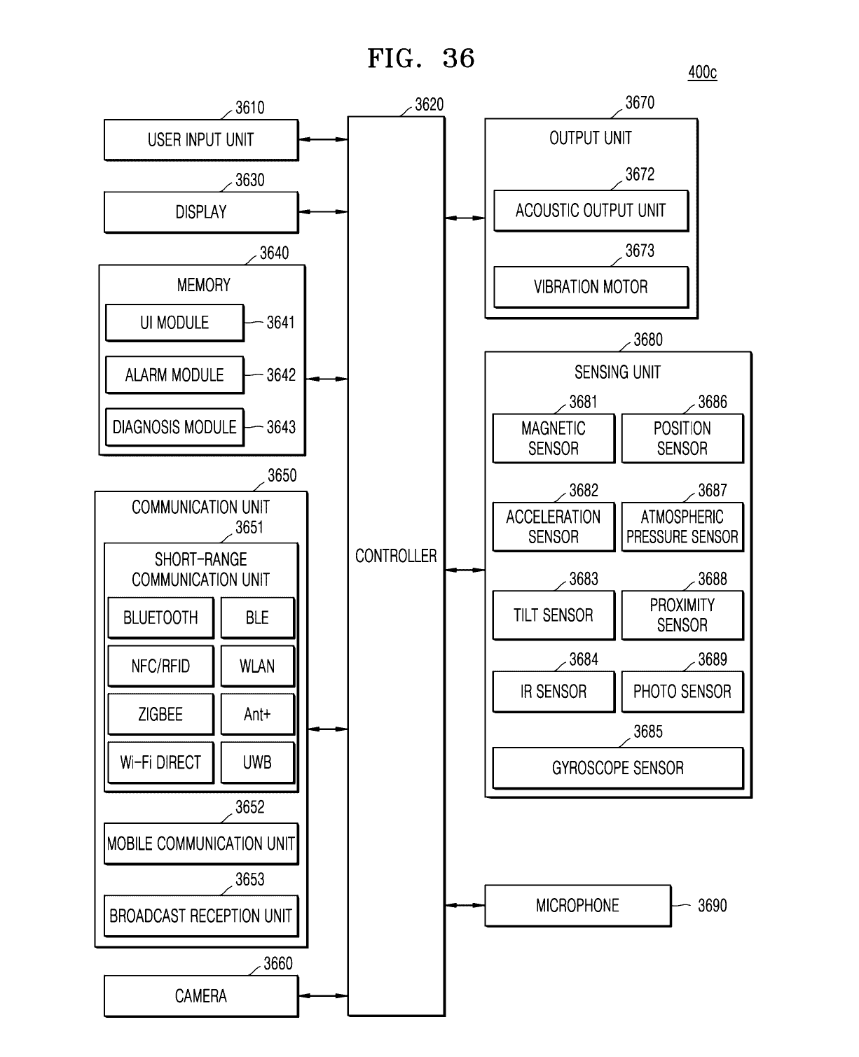

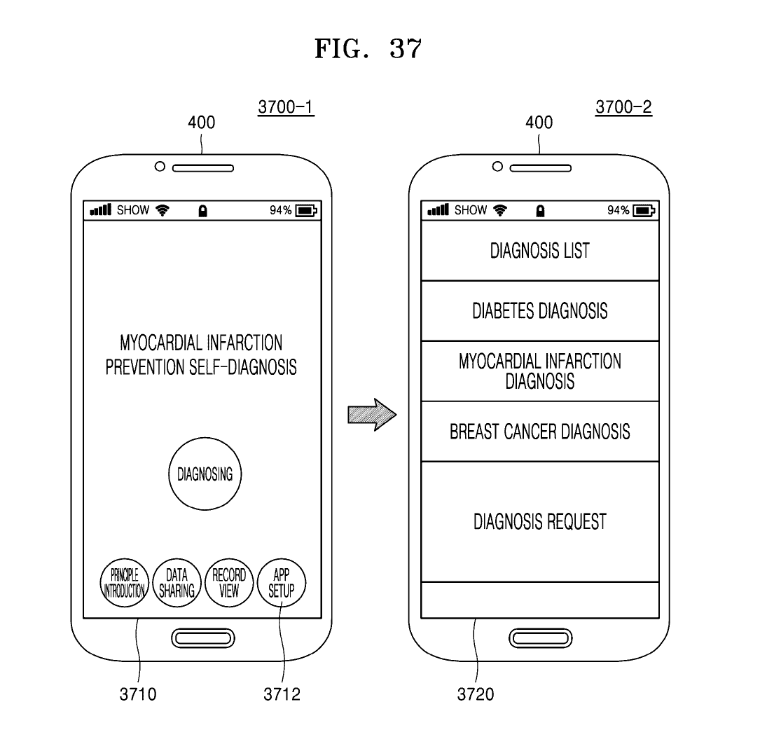

FIG. 36 illustrates a block diagram of an analysis apparatus which may perform functions besides a diagnosis application, according to an embodiment of the present disclosure.

Referring to FIG. 36, an analysis apparatus 400c may include a user input unit 3610 configured to receive user input. For example, the user input unit 3610 may include a keypad, a dome switch, a touch pad (a capacitive overlay touch pad, a resistive overlay touch pad, an IR beam touch pad, a surface acoustic wave touch pad, an integral strain gauge touch pad, a piezoelectric touch pad, or the like), a jog wheel, a jog switch, and the like but is not limited thereto.

The user input unit 3610 may receive a user input for executing a diagnosis application. According to an embodiment, the user input for executing the diagnosis application may be various. For example, the user input may include a key input, a touch input, a motion input, a bending input, a voice input, a multi-input, and the like.

A controller 3620 may commonly control a general operation of the analysis apparatus 400c. For example, the controller 3620 may generally control the user input unit 3610, an output unit 3670, a communication unit 3650, a sensor 3680, a microphone 3690, and the like by executing programs stored in a memory 3640.

A display 3630 may display information processed by the analysis apparatus 400c. For example, the display 3630 may display a still image, a moving picture, a live-view image, and the like. When the display 3630 and a touch pad form a layer structure to configure a touch screen, the display 3630 may be used as not only an output device but also an input device. The display 3630 may include at least one of a liquid crystal display, a thin-film transistor liquid crystal display, an organic LED (OLED), a flexible display, a three-dimensional (3D) display, and an electrophoretic display. The analysis apparatus 400c may include two or more displays 3630 according to an implementation form of the analysis apparatus 400c.

The memory 3640 may store programs for processing and control of the controller 3620 and store inputted/outputted data (e.g., a plurality of images, a plurality of folders, and a preferred folder list).

The memory 3640 may include at least one type of storage medium among a flash memory type memory, a hard disk type memory, a multimedia card micro type memory, a card type memory (e.g., a secure digital (SD) or extreme digital (XD) memory), RAM, static RAM (SRAM), ROM, electrically erasable programmable ROM (EEPROM), PROM, a magnetic memory, a magnetic disc, and an optical disc. In addition, the analysis apparatus 400c may operate a cloud storage which performs a storage function via the Internet.

The programs stored in the memory 3640 may be classified into a plurality of modules according to functions thereof, e.g., a user interface (UI) module 3641, an alarm module 3642, a diagnosis module 3643, and the like.

The UI module 3641 may provide a specified UI, a graphical user interface (GUI), or the like interoperating with the analysis apparatus 400c for each application. The alarm module 3642 may generate a signal for notifying of the occurrence of an event of the analysis apparatus 400c. The alarm module 3642 may output an alarm signal in a video signal form through the display 3630, an alarm signal in an audio signal form through an acoustic output unit 3672, or an alarm signal in a vibration signal form through a vibration motor 3673.

The diagnosis module 3643 may acquire target material information from optical information acquired from a strip for analysis. In addition, the diagnosis module 3643 may acquire health state information from the target material information. The controller 3620 may perform the same functions as the processor 2420, 3020, or 3420 described above by executing programs stored in the diagnosis module 3643. Hereinafter, for convenience of description, it is assumed that the diagnosis module 3643 executes a diagnosis application. For example, the diagnosis module 3643 may acquire target material information based on the optical information by using a lookup table in which the optical information matches target material information.

When the target material information is acquired, the diagnosis module 3643 may acquire the target material information by using an amount of a sample introduced into the analysis strip 100, user information (e.g., age, sex, and medical record information), a geographical location where a diagnosis was performed, a time that the diagnosis was performed, information related to the strip (e.g., a manufacturer and a manufacturing date of the strip), and the like.

In addition, the diagnosis module 3643 may acquire target material information by using first optical information detected from the target strip 20 and second optical information detected from the auxiliary strip 30. For example, when the analysis strip 100 according to an embodiment includes one target strip 20 and two auxiliary strips 30 having different densities of a reference material, the diagnosis module 3643 may acquire target material information by using one piece of first optical information acquired from the one target strip 20 and two pieces of second optical information acquired from the two auxiliary strips 30.

The controller 3620 may determine whether a contained percentage of a target material is normal or abnormal based on the acquired target material information and provide the determination result, i.e., a diagnosis result, to a user through the display 3630. The controller 3620 may provide a user guide according to the diagnosis result. For example, the controller 3620 may provide a user guide “clinical treatment required.” In addition, the user guide may include exercise habits, eating habits, sleeping habits, and the like.

The communication unit 3650 may include one or more components enabling the analysis apparatus 400c to communicate with a cloud server, an external device, a social networking service (SNS) server, or an external wearable device. For example, the communication unit 3650 may include a short-range communication unit 3651, a mobile communication unit 3652, and a broadcast reception unit 3653.

The short-range wireless communication unit 3651 may include a Bluetooth communication unit, a Bluetooth low energy (BLE) communication unit, a near-field communication (NFC) unit, a wireless local area network (WLAN) Wi-Fi communication unit, a Zigbee communication unit, an infrared data association (IrDA) communication unit, a Wi-Fi direct (WFD) communication unit, an ultra-wideband (UWB) communication unit, an Ant+ communication unit, and the like but is not limited thereto.

The mobile communication unit 3652 may transmit and receive a wireless signal to and from at least one of a base station, an external terminal, and a server in a mobile communication network. Herein the wireless signal may include a voice call signal, a video call signal, or various types of data according to text/multimedia message transmission and reception.

The broadcast reception unit 3653 may receive a broadcast signal and/or broadcast related information from the outside through a broadcast channel, and the broadcast channel may include a satellite channel and a terrestrial channel. According to implemented examples, the analysis apparatus 400c may not include the broadcast reception unit 3653.

The communication unit 3650 may share optical information, health state information, and the like with an external device. The external device may be any one of a cloud server, an SNS server, another analysis apparatus of the same user, and an analysis apparatus of another user, which are connected to the analysis apparatus 400c but is not limited thereto.

A camera 3660 may detect light which is reflected from or has transmitted through the analysis strip 100. The detected optical information may be stored in the memory 3640 or transmitted to the outside via the communication unit 3650. Two or more cameras 3660 may be provided according to a configuration aspect.

The output unit 3670 is configured to output an audio signal, a video signal, or a vibration signal via one or more of the display 3630, the acoustic output unit 3672, and the vibration motor 3673.

The acoustic output unit 3672 may output audio data received through the communication unit 3650 or stored in the memory 3640. In addition, the acoustic output unit 3672 may output an acoustic signal related to a function (e.g., a call signal reception sound, a message reception sound, or an alarm sound) performed by the analysis apparatus 400c. The acoustic output unit 3672 may include a speaker, a buzzer, and the like.

The vibration motor 3673 may output a vibration signal. For example, the vibration motor 3673 may output a vibration signal corresponding to an output of audio data or video data (e.g., a call signal reception sound, or a message reception sound). In addition, the vibration motor 3673 may output a vibration signal when a touch is inputted through the touch screen.

The sensor 3680 may detect a state of the analysis apparatus 400c, a state of the surroundings of the analysis apparatus 400c, a state of a user wearing the analysis apparatus 400c, and the like and transmit the detected information to the controller 3620.

The sensor 3680 may include at least one of a magnetic sensor 3681, an acceleration sensor 3682, a tilt sensor 3683, an IR sensor 3684, a gyroscope sensor 3685, a position sensor (e.g., global positioning system (GPS) sensor) 3686, an atmospheric pressure sensor 3687, a proximity sensor 3688 and a photo sensor 3689 but is not limited thereto. The sensor 3680 may also include a temperature sensor, an illumination sensor, a pressure sensor, an iris recognition sensor, and the like. A function of each sensor may be intuitively inferred by those of ordinary skill in the art from a name thereof, and thus a detailed description thereof is omitted herein.

A microphone 3690 may be included as the audio/video input unit.

The microphone 3690 may receive an external acoustic signal and process the external acoustic signal to electrical voice data. For example, the microphone 3690 may receive an acoustic signal from an external device or a speaker. The microphone 3690 may use various noise cancellation algorithms to cancel noise generated during a process of receiving an external acoustic signal.

Hereinafter, an operation of an analysis apparatus related to execution of a diagnosis application. The analysis apparatus 400c according to an embodiment may include a setup mode in which a user sets up a plan for a diagnosis, a diagnosis mode for analyzing target material information and providing the analysis result, and a sharing mode for sharing the analysis result with an external device.

FIG. 37 illustrates a diagram for describing a setup mode of the diagnosis application according to an embodiment of the present disclosure.

Referring to FIG. 37, an analysis apparatus 400 may download a diagnosis application through an application store (e.g., an external server) according to a user input. Alternatively, when a user buys the analysis apparatus 400, the diagnosis application may be pre-stored in the analysis apparatus 400. The analysis apparatus 400 may execute the diagnosis application according to a user input.

Once the diagnosis application is executed, the analysis apparatus 400 may display an initial screen image 3710 as shown in reference numeral 3700-1. The initial screen image 3710 may include various kinds of information for a diagnosis by a GUI or the like.

For example, the initial screen image 3710 may include a principle introduction item for describing the diagnosis application, a setup item 3712 for setting up detailed items, a record view item for providing a result according to a diagnosis of the diagnosis application, and a sharing item for sharing the result of the diagnosis application with an external device.

The user may input a user command for selecting any one of the items described above. For example, if the user inputs a command for selecting the setup item 3712, the analysis apparatus 400 may provide various items related to settings. For example, as shown in reference numeral 3700-2, a diagnosis list 3720 lists various diagnosis types. The diagnosis list 3720 may include diabetes diagnosis, myocardial infarction diagnosis, and breast cancer diagnosis, for example. The user may input a command for selecting at least one diagnosis item of the diagnosis list 3720.

Although 3700-2 of FIG. 37 shows that the analysis apparatus 400 displays the diagnosis list 3720, the present embodiment is not limited thereto. If the diagnosis application is an application for a certain diagnosis item, the analysis apparatus 400 may not display the diagnosis list 3720. For example, if the diagnosis application is an application for diagnosing myocardial infarction, the analysis apparatus 400 may not display the diagnosis list 3720.

Although it has been described with reference to FIG. 37 that the analysis apparatus 400 determines a diagnosis item according to the user's direct input, the present embodiment is not limited thereto. The user may directly select a diagnosis item, or when user information is inputted, the analysis apparatus 400 may determine a diagnosis item based on the user information.

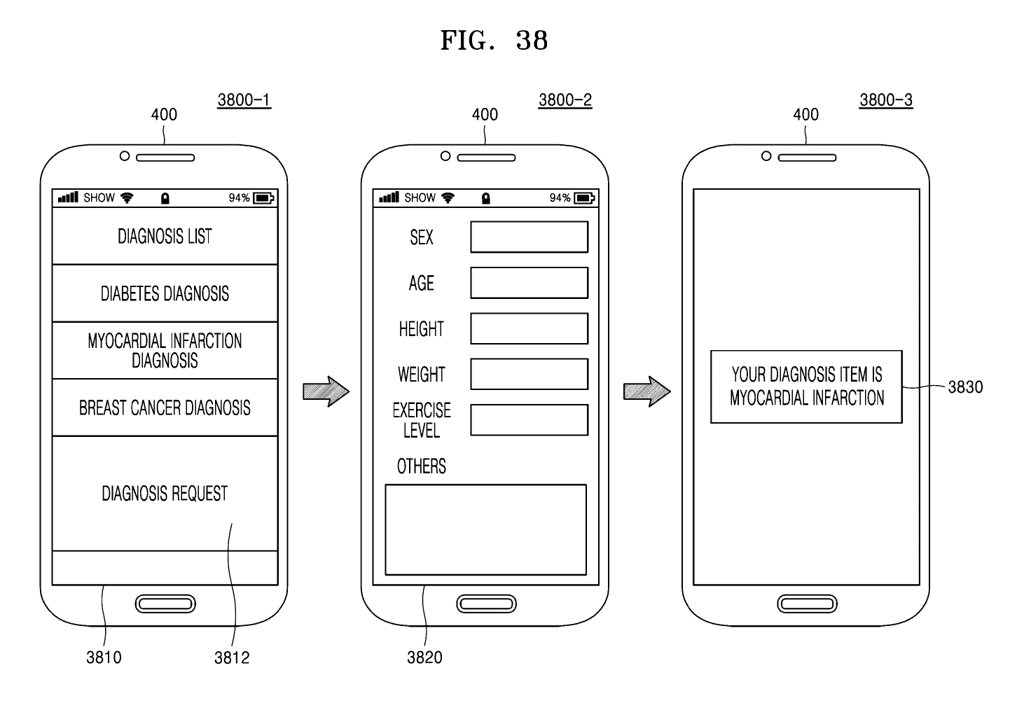

FIG. 38 illustrates a diagram for describing a method of determining a diagnosis item according to an embodiment of the present disclosure.

Referring to FIG. 38, in reference numeral 3800-1, a diagnosis list 3810 may include a diagnosis request item 3812 for the analysis apparatus 400 to determine a diagnosis item based on user information.

If a user inputs a command for selecting the diagnosis request item 3812, the analysis apparatus 400 may provide a screen image 3820 for inputting user information as shown in reference numeral 3800-2. The user may input various pieces of user information in a GUI. The user information may include, for example, age, sex, height, weight, exercise level, smoking, drinking capacity, medical history, and family history.

Referring to reference numeral 3800-3, the analysis apparatus 400 may determine and provide a diagnosis item requesting for a diagnosis by using the inputted user information. For example, if the user is a 65-year old male, has an experience of an emergency treatment due to cardiac arrest two weeks ago, had smoked for 25 years, and does not usually exercise, the analysis apparatus 400 may determine that the user has the risk of getting a disease “myocardial infarction,” determine myocardial infarction as a diagnosis item, and display a result 3630 of the determination.

As another example, if the user is a 25-year old female, is overweight, has a high carbohydrate intake, and has a family history of diabetes, the analysis apparatus 400 may determine that the user has the risk of “diabetes” and determine diabetes as a diagnosis item.

To determine a diagnosis item, the analysis apparatus 400 may use a database including a correlation between age, sex, eating habits, exercise habits, and the like and a disease. The database may be pre-stored in the analysis apparatus 400 or stored in an external device. If the database is stored in the external device, the analysis apparatus 400 may communicate with the external device and use the database.

The analysis apparatus 400 may determine one diagnosis item for requesting the user to be diagnosed, by using inputted user information. However, the present embodiment is not limited thereto. The analysis apparatus 400 may determine a plurality of diagnosis items by using inputted user information.

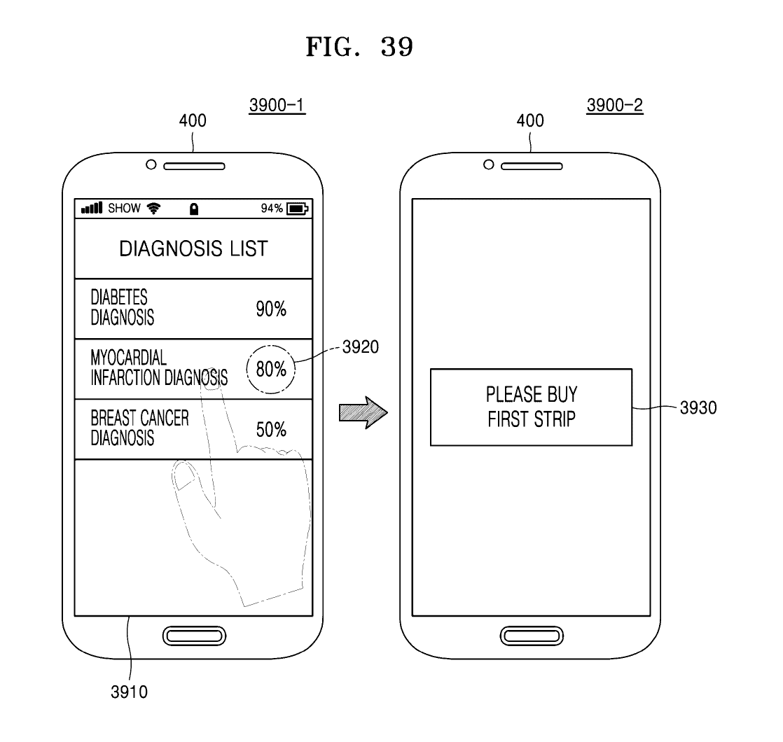

FIG. 39 illustrates a reference diagram for describing a method of providing a diagnosis item, according to an embodiment of the present disclosure.

Referring to FIG. 39, reference numeral 3900-1, illustrates that the analysis apparatus 400 may provide a diagnosis list 3910 including a plurality of diagnosis items requesting for a diagnosis. In this case, the analysis apparatus 400 may also provide priority 3920 indicating an item preferentially requesting for a diagnosis. The user may select any one item of the diagnosis list 3910. In response to the selection, the analysis apparatus 400 may provide analysis strip information 3930 corresponding to the selected diagnosis item as shown in reference numeral 3900-2. Since a target material depends on a diagnosis item, a type of an analysis strip may vary in correspondence with the target material.

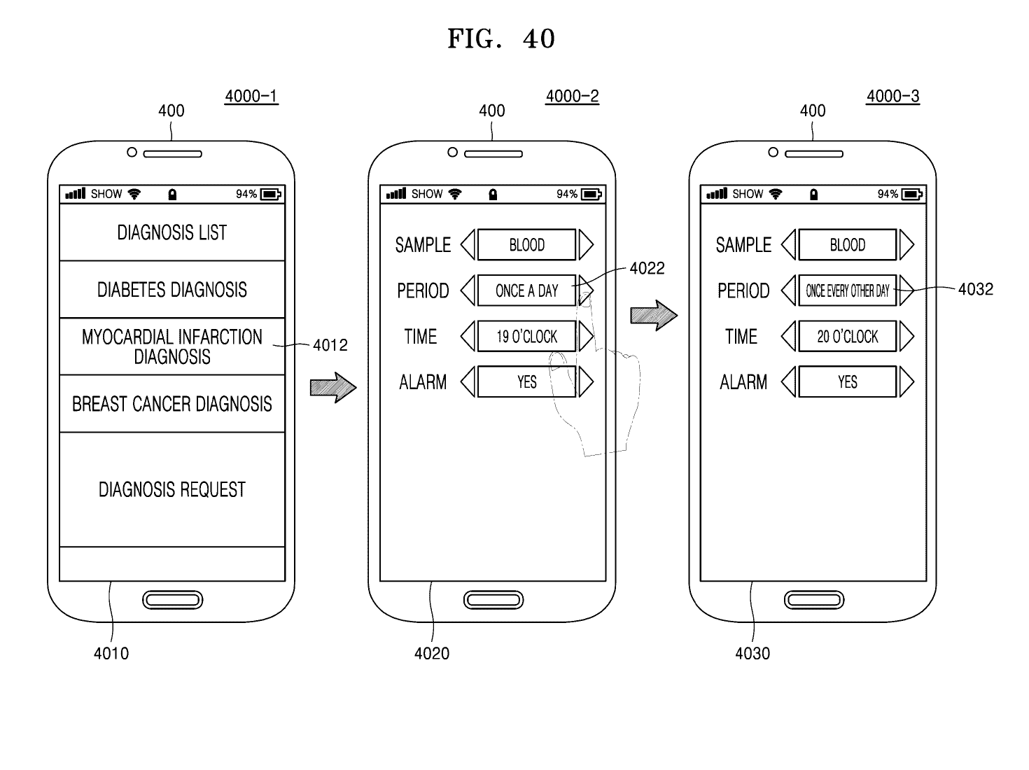

FIG. 40 illustrates a diagram for describing a method of setting a detailed plan of a diagnosis item, according to an embodiment of the present disclosure.

Referring to FIG. 40, if a diagnosis item is determined, the analysis apparatus 400 may provide a screen image 4010 for setting a detailed plan of the determined diagnosis item as shown in reference numeral 4000-1. For example, if the user inputs a command for selecting a diagnosis item 4012 “myocardial infarction”, the analysis apparatus 400 may provide a screen image 4020 for a detailed plan of the diagnosis item 4012 as shown in reference numeral 4000-2.

For example, the detailed plan of myocardial infarction may include a type of a sample for a diagnosis, a diagnosis period, a diagnosis time, an alarm on whether the diagnosis time is notified, and the like. An initial detailed plan provided by the analysis apparatus 400 may be a preset detailed plan but is not limited thereto. If the initial detailed plan is not preset, the analysis apparatus 400 may provide only items according to the detailed plan. Thereafter, the analysis apparatus 400 may determine a detailed plan according to a user input.

In addition, even though the initial detailed plan is preset, the analysis apparatus 400 may change the detailed plan according to a user input. For example, as shown in reference numeral 4000-2, the user may input a command 4022 for changing at least one item of the detailed plan. The analysis apparatus 400 may display a detailed plan 4030 including an item 4032 changed according to the user's input as shown in reference numeral 4000-3.

A detailed plan adjustment range of the user may be within a range by which a health state of the user is determined based on a diagnosis result.

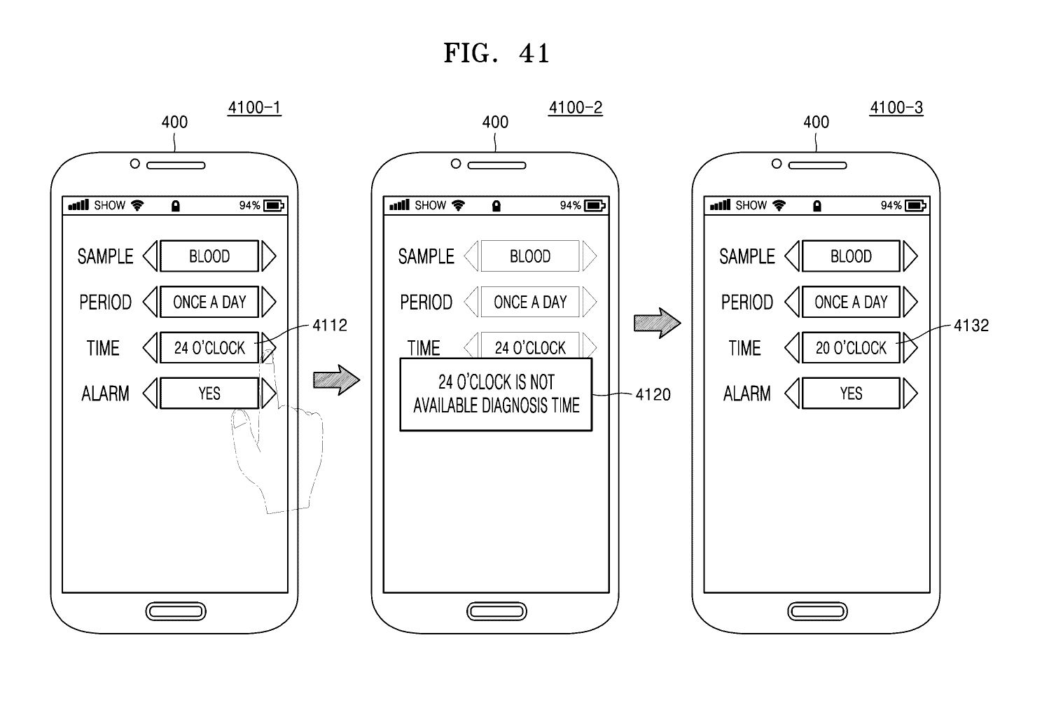

FIG. 41 illustrates a diagram for describing a method of changing a detailed plan, according to an embodiment of the present disclosure.

Referring to FIG. 41, in reference numeral 4100-1, if a user input 4112 for changing a detailed plan is received, the analysis apparatus 400 may determine whether the changed detailed plan corresponds to data by which a health state is determined. For example, when the user changes a diagnosis time to 24 o'clock, the analysis apparatus 400 determines whether a diagnosis result at 24 o'clock corresponds to data by which myocardial infarction or not is determined. If it is determined that the diagnosis result at 24 o'clock does not correspond to valid data, the analysis apparatus 400 may provide an alarm 4120 indicating that the diagnosis time is not available as shown in reference numeral 4100-2. Alternatively, the analysis apparatus 400 may determine the diagnosis plan with a boundary value 4132 within a changeable range. For example, even though the user sets a diagnosis time as 24 o'clock, the analysis apparatus 400 may determine the diagnosis plan as 20 o'clock as shown in 4100-3 of FIG. 41.

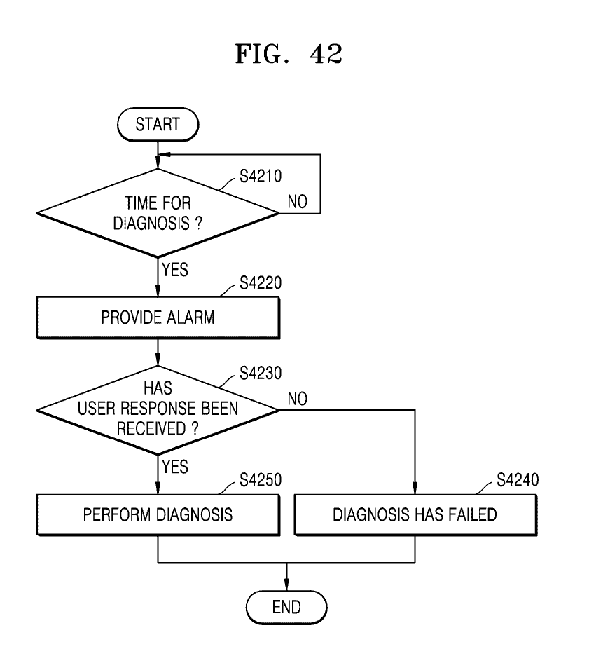

FIG. 42 is a flowchart of a method for executing a diagnosis according to an embodiment of the present disclosure.

Referring to FIG. 42, the analysis apparatus 400 may determine if it is time for a diagnosis in operation S4210. If is it not time for a diagnosis, the method continues to wait until it is time for diagnosis in operation S4210. When it is time for the diagnosis, an alarm is output in operation S4220. The analysis apparatus 400 may determine whether a current time point is a diagnosis time included in a detailed plan of a diagnosis item and provide an alarm for a diagnosis if the diagnosis time is in time. The alarm may be provided using an image, a text, an audible frequency, vibrations, emission of light, a temperature change, a pressure change, or the like.

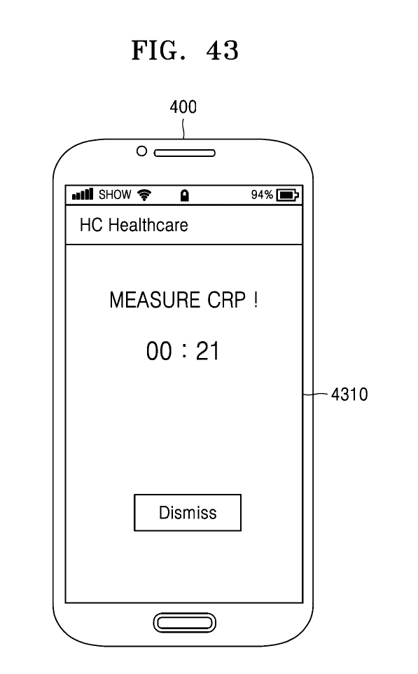

FIG. 43 illustrates a reference diagram for describing an alarm for a diagnosis, according to an embodiment of the present disclosure.

Referring to FIG. 43, the analysis apparatus 400 may provide an alarm 4310 with a text on a display. However, the present embodiment is not limited thereto. The provision of an alarm may be omitted according to a user's selection.

Referring back to FIG. 42, the analysis apparatus 400 may determine whether a user response responding to a diagnosis has been received in operation S4230. For example, if a diagnosis item is myocardial infarction, the user may collect blood of the user and introduces the collected blood into the introduction part 10 of the analysis strip 100. Thereafter, the user may insert the analysis strip 100 into a system for analysis and activate at least one of the analysis reader 300 and the analysis apparatus 400. Alternatively, once the analysis strip 100 is inserted into the analysis reader 300, the analysis reader 300 may be automatically activated, and the analysis reader 300 may transmit a diagnosis result to the analysis apparatus 400. Alternatively, if the analysis system is implemented only with the analysis apparatus 400, when the user places the analysis strip 100 at a location where the analysis apparatus 400 is detectable, the analysis apparatus 400 may detect the analysis strip 100 and be automatically activated.

Through any one of the user's behaviors, the analysis apparatus 400 may receive a user response. For example, when the analysis strip 100 is inserted into the analysis reader 300, and optical information is received from the analysis reader 300, it may be determined that the analysis apparatus 400 has received a user response. Alternatively, the user may input a user response by inserting the analysis strip 100 into a system for analysis and then selecting a certain key of the analysis apparatus 400. In this case, the analysis apparatus 400 may determine that the user response has been received and control the analysis reader 300 or the analysis apparatus 400 to acquire target material information.

If no user response has been received in operation 54230, the analysis apparatus 400 determines that the diagnosis has failed in operation 54240. Thereafter, the analysis apparatus 400 may provide or record the determination result.

Otherwise, if the user response has been received in operation S4230, the analysis apparatus 400 may perform a diagnosis in operation 54250. For example, if optical information is received from the analysis reader 300, the analysis apparatus 400 may acquire target material information by using the optical information. When the target material information is acquired, first optical information acquired from the target strip 20 and second optical information acquired from the auxiliary strip 30. Alternatively, when the analysis apparatus 400 includes an image sensor capable of photographing the analysis strip 100, the analysis apparatus 400 may acquire optical information by receiving light outputted from the analysis strip 100 and acquire target material information.

The analysis apparatus 400 may provide and record a diagnosis result. The analysis apparatus 400 may provide a diagnosis result every time a diagnosis is performed. The analysis apparatus 400 may display target material information as a value, or determine a health state based on acquired target material information and then provide the determination result.

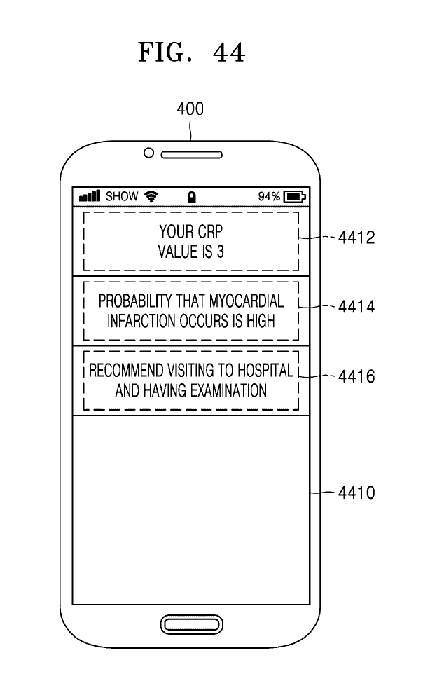

FIG. 44 illustrates a diagram for describing a method of providing a diagnosis result according to an embodiment of the present disclosure.

Referring to FIG. 44, the analysis apparatus 400 may display a screen image 4410 for a diagnosis result. The screen image 4410 for a diagnosis result may include target material information 4412, health state information 4414 according to the target material information 4412, and the like. In addition, the analysis apparatus 400 may provide a user guide 4416 according to the diagnosis result. For example, a user guide “clinical treatment required” may be provided.

Although not shown, if a user command for selecting a user guide is inputted, the analysis apparatus 400 may provide a GUI through which a treatment may be reserved by searching the Internet or the like for a hospital, a treatment department, and the like related to the diagnosis result. Alternatively, the analysis apparatus 400 may automatically reserve a preset hospital for a treatment.

When a health state according to a diagnosis result is within a reference range, e.g., a normal range, the analysis apparatus 400 may not display the diagnosis result because the user may be interest in only a range which may badly affect a health state without being interested in every diagnosis result. Therefore, display of a diagnosis result may be determined by a selection of the user.

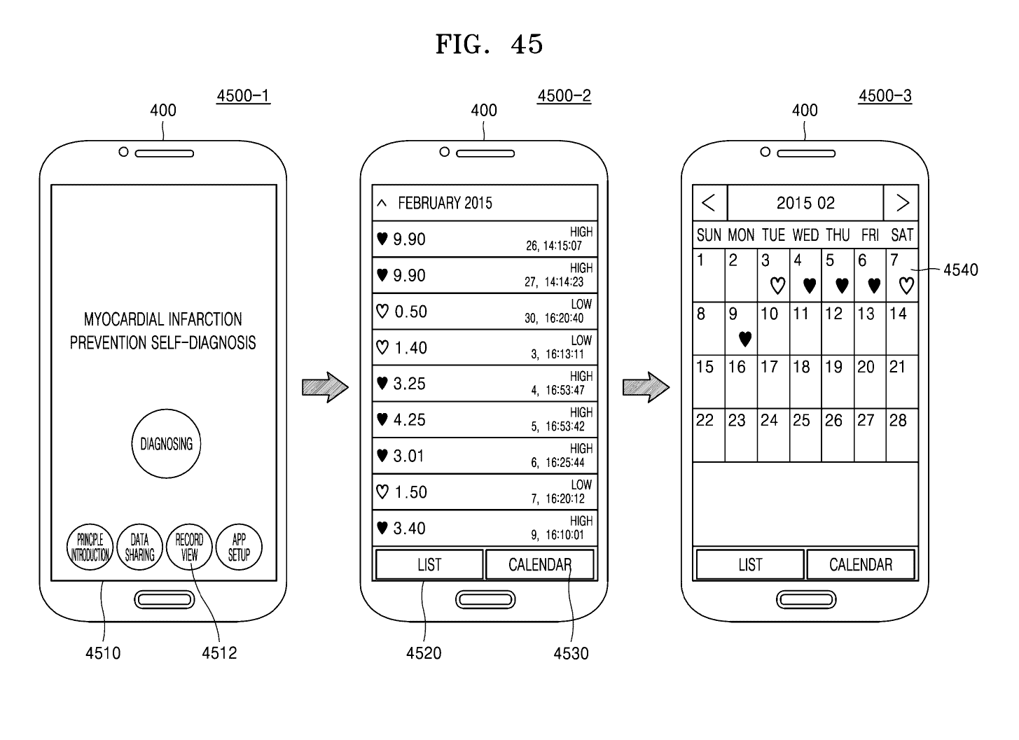

FIG. 45 illustrates a reference diagram for describing a method of providing a diagnosis result, according to an embodiment of the present disclosure.

Referring to FIG. 45, in a screen image 4510 shown in reference numeral 4500-1, the user may input a command for selecting a record view item 4512. In this case, the analysis apparatus 400 may sequentially provide pre-recorded diagnosis results 4520 as shown in reference numeral 4500-2. If a user command for selecting a calendar item 4530 is received, the analysis apparatus 400 may provide diagnosis results 4540 in a calendar type as shown in reference numeral 4500-3. If a user command for selecting a certain item among the diagnosis results 4540 is received, the analysis apparatus 400 may provide a GUI through which detailed information of the selected item may be provided or edited.

A diagnosis result may be shared with an external device. As described above, when a treatment is reserved according to a diagnosis result, the diagnosis result may be transmitted to a corresponding to a medical institution by a short message service (SMS) type or the like. A medical service may be provided by various methods besides the method described above.

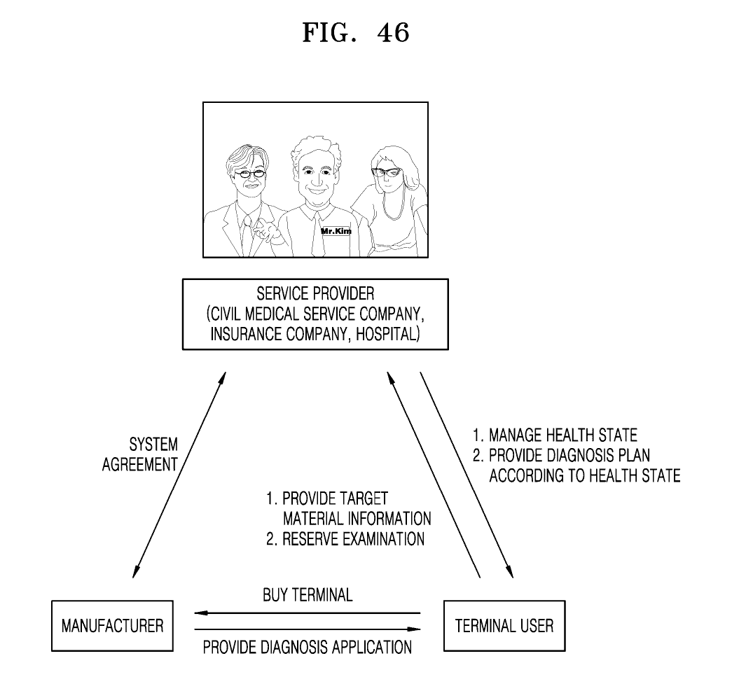

FIG. 46 illustrates a diagram for describing a network supporting a medical service according to an embodiment of the present disclosure.

Referring to FIG. 46, the network may be configured with a manufacturer of the analysis system for acquiring information related to a target material included in a sample, a service provider for providing the medical service, and a terminal user using the medical service.

The manufacturer of the analysis system may manufacture the analysis for analysis 100, the analysis reader 300, and the analysis apparatus 400. Alternatively, the manufacturer of the analysis system may manufacture only the analysis for analysis 100 and the analysis apparatus 400 or only the analysis apparatus 400.

The service provider may include medical institutions, such as a hospital and a pharmacy, and organizations, such as an insurance company, which request for health state information of a user. It is recommended that a network system store various information related to target materials. The service provider may manufacture the analysis for analysis 100 enabling point-of-care testing (POCT).

The terminal user (herein, the terminal user may be a user of the analysis apparatus 400) is a subject receiving the medical service and may collect a sample such as urine or blood of the terminal user and perform measurement by using a diagnosis application (e.g., S-health) of a terminal. The terminal user may form an offline/online contractual relationship (e.g., a subscription) with the service provider. For example, the terminal user may be a patient and the service provider may be a hospital where the terminal user was an inpatient.

If it is difficult for the terminal user to visit the hospital or only very simple measurement is requested, measurement using a terminal may be performed, and thus, the hospital which is the service provider may send the analysis strip 100 to the terminal user. As another example, the terminal user may be a subject desiring to subscribe to insurance and the service provider may be an insurance company. The terminal user needs to measure and transmit a health state (e.g., target material information) of the terminal user for the purpose of subscription to insurance, and thus, the insurance company may send the analysis strip 100 to the terminal user.

The terminal user may measure a health state of the terminal user by using the diagnosis application of the terminal and transmit the measurement result to the service provider, and the service provider may store the measurement result of the terminal user. Since the health state of the terminal user is stored by the service provider and the terminal user, information related to a treatment of various diseases may be shared.

The service provider may periodically or aperiodically provide the medical service to the terminal user to manage the health state of the terminal user. A service for managing a real-time health state of the terminal user in addition to physical information measured by a wearable device such as a smart watch or smart glasses may be provided to the terminal user.

It should be understood that embodiments described herein should be considered in a descriptive sense only and not for purposes of limitation. Descriptions of features or aspects within each embodiment should typically be considered as available for other similar features or aspects in other embodiments.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. An analysis strip comprising:
an introduction part through which a sample is introduced;
a target strip connected to the introduction part and configured to detect concentration of a target material included in the sample according to a flow of the introduced sample; and
an auxiliary strip connected to the introduction part that is pre-impregnated with certain densities and configured to detect concentration of a mixed material of the target material and the pre-impregnated material according to the flow of the introduced sample,
wherein the target strip and the auxiliary strip comprise a same material.

2. The analysis strip of claim 1, wherein the auxiliary strip comprises a first auxiliary strip having a first density and a second auxiliary strip having a second density.

3. The analysis strip of claim 2, wherein the first and second auxiliary strips are spaced apart from each other by interposing the target strip therebetween.

4. The analysis strip of claim 2, wherein a location of the pre-impregnated material in the first auxiliary strip corresponds to a location of the pre-impregnated material impregnated in the second auxiliary strip.

5. The analysis strip of claim 1, wherein each of the target strip and the auxiliary strip has a conjugating region having a conjugating body bondable with the target material is impregnated.

6. The analysis strip of claim 5, wherein the pre-impregnated material is impregnated between the introduction part and the conjugating region.

7. The analysis strip of claim 1, wherein each of the target strip and the auxiliary strip includes a test region for detecting the target material.

8. The analysis strip of claim 7, wherein a location of the test region in the target strip corresponds to a location of the test region in the auxiliary strip.

9. The analysis strip of claim 7, wherein the pre-impregnated material is impregnated between the introduction part and the test region.

10. The analysis strip of claim 1, wherein the test region is color-developed by the target material.

11. The analysis strip of claim 1, further comprising an absorption part configured to absorb the sample,
wherein a distal end of the target strip and a distal end of the auxiliary strip are connected to the absorption part.

12. An analysis apparatus comprising:
a light-receiver configured to receive optical information from an analysis strip comprising a target strip and first and second auxiliary strips having a target material pre-impregnated with different densities; and
a controller configured to determine target material information based on the optical information, a first information of the target materials pre-impregnated in the first auxiliary strip, and a second information of the target materials pre-impregnated in the second auxiliary strip.

13. The analysis apparatus of claim 12, wherein the optical information includes first optical information corresponding to the target strip, second optical information corresponding to the first auxiliary strip, and third optical information corresponding to the second auxiliary strip.

14. The analysis apparatus of claim 13, wherein the controller is further configured to determine the target material information based on the optical information and a lookup table.

15. The analysis apparatus of claim 13, wherein the controller is further configured to determine the target material information related to the target material by using a difference between the second optical information and the third optical information, a difference between the first information the second information, and the first optical information.

16. The analysis apparatus of claim 15, wherein the target material information is obtained by dividing a product of the difference between the first information and the second information about and the first optical information by the difference between the second optical information and the third optical information.

17. The analysis apparatus of claim 12, further comprising an output unit configured to output the target material information.

18. The analysis apparatus of claim 17, wherein the output unit is further configured to output the information of the target material as a text or a graph.

19. The analysis apparatus of claim 12, further comprising a light source configured to emit light onto the analysis strip.

20. A target material analysis method comprising:
acquiring optical information from an analysis strip comprising a target strip and an auxiliary strip having a pre-impregnated target material; and
acquiring target material information of the target material by using the optical information and first information related the target material pre-impregnated in the auxiliary strip.
